SEQUENCE LISTING (1 sequence)

SEQ ID NO: 1 - DNA, 12010 bp, Artificial Sequence
  Description of Artificial Sequence Synthetic polynucleotide
acgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatccggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgcgactcca

(19) United States
(12) Patent Application Publication
(10) Pub. No.: US 2023/0159950 A1
(43) Pub. Date: May 25, 2023

(54) METHODS AND COMPOSITIONS FOR PRODUCTION OF RECOMBINANT ADENO-ASSOCIATED VIRUSES

(71) Applicant: Biogen MA Inc., Cambridge, MA (US)

(72) Inventors: Bryan Joseph St. Germain, Medford, MA (US); Michael James Volles, Cambridge, MA (US)

(21) Appl. No.: 17/912,087

(22) PCT Filed: Mar. 17, 2021
(86) PCT No.: PCT/US2021/022854

§ 371 Date: Sep. 16, 2022

Related U.S. Application Data
(60) Provisional application No. 62/990,531, filed on Mar. 17, 2020.

(51) Int. Cl. C12N 15/86

(52) U.S. Cl. CPC C12N 15/86; C12N 2750/14122; C12N 2750/14143; C12N 2750/14152; C12N 2710/10344; C12N 2710/10322

(57) ABSTRACT

The present disclosure pertains to compositions and methods for the production of recombinant adeno-associated viruses (rAAVs).

### BACKGROUND

Recombinant adeno-associated viruses (rAAVs) are engineered by deleting, in whole or in part, the internal portion of an AAV genome and inserting a heterologous nucleic acid of interest between inverted terminal repeats (ITRs). ITRs remain functional in such vectors (e.g., plasmids), thereby allowing for replication and packaging of the rAAV particles containing the payload enclosed within the AAV capsid. A major challenge in the development of rAAV as a gene therapy is manufacturing technologies that yield the quantity and quality of rAAV particles needed for clinical applications.

Thus, there remains a need for improved methods and compositions for manufacturing rAAV particles. In particular, there is a need for methods and compositions that can be used to efficiently produce large quantities of rAAVs for gene therapy products.

### SUMMARY

The present description encompasses, inter alia, methods and compositions for producing a plurality of recombinant adeno-associated virus (rAAV) particles. The rAAV particles can have one or more improved characteristics relative to a reference vector (e.g., an Ad5 helper vector). Such improved characteristics relative to a reference vector can include, but are not limited to, one, two, three, four, five, six, seven, eight, or nine of: (i) improved infectivity, (ii) increased expression or amount of a payload, (iii) improved transduction, (iv) higher titer (e.g., AAV titer) production, (v) lower amount of helper vector required to produce rAAV particles, (vi) greater purity of rAAV particles (e.g., reduced adenoviral contamination), (vii) larger scale production of rAAV, (viii) improved formation of transfection complexes, or (ix) greater yield of helper vector produced in host cells (e.g., bacterial cells, e.g., E. coli cells).

In one aspect, the disclosure provides methods of producing a plurality of recombinant adeno-associated virus (rAAV) particles comprising: (a) transfecting a host cell with (i) a first vector encoding at least one Adenovirus 2 (Ad2) helper polypeptides; (ii) a second vector encoding at least one Rep polypeptide and/or at least one Cap polypeptide; and (iii) a third vector encoding at least one payload; and (b) culturing a host cell under conditions suitable for production of a plurality of rAAV particles, thereby producing a plurality of rAAV particles.

In some embodiments, Ad2 helper polypeptides comprise at least one of E1, E2A, E4, or VA RNA. In some embodiments, Ad2 helper polypeptides comprise at least two of E1, E2A, E4, or VA RNA. In some embodiments, Ad2 helper polypeptides comprise at least three of E1, E2A, E4, or VA RNA. In some embodiments, Ad2 helper polypeptides comprise all four of E1, E2A, E4, or VA RNA. In some embodiments, a Cap polypeptide comprises an AAV1, AAV2, AAV3, AAV4, AAV5, AAV6, AAV7, AAV8, AAV9, AAV10, or AAV11 Cap polypeptide, or a variant of any of the foregoing. In some embodiments, a Rep polypeptide comprises an AAV2 Rep polypeptide, or a variant thereof.

In some embodiments, host cells are or comprise adherent cells. In some embodiments, host cells are or comprise suspension cells.

In some embodiments, a plurality of rAAV particles are produced in a large-scale preparation. In some embodiments, a large-scale preparation is at least 10 liters+/−15% of culture media. In some embodiments, a large-scale preparation is at least 5 m²+/−15% of culture media. In some embodiments, host cells are transfected using polyethylenimine (PEI). In some embodiments, transfection complexes are formed for less than 10 minutes+/−15%. In some embodiments, host cells were seeded prior to a transfecting step at a density of: at least 1.0×10⁴ viable cells (vc)/cm²+/−15%, or at least 1.0×10⁵ vc/cm²+/−15%.

In some embodiments, a host cell is a mammalian cell. In some embodiments, a mammalian cell is selected from HEK293 cells, CHO-K, or HeLa cells. In some embodiments, a host cell expresses an E1 polypeptide. In some embodiments, the host cell does not express an E1 polypeptide.

In some embodiments, a plurality of AAV particles are produced at a high titer. In some embodiments, a high titer is relative to AAV particles produced from an Ad5 helper vector under otherwise identical conditions. In some embodiments, a high titer is greater than: (i) 2.6×10¹⁰ viral genomes (vg)/cm²+/−15% when cultured in a flask. In some embodiments, a high titer is greater than: (i) 6.3×10⁹ vg/mL²+/−15% when cultured in an adherent culture, e.g., a fixed bed bioreactor or a flask; (ii) 2.6×10¹⁰ vg/cm²+/−15% when cultured in a flask; and/or (ii) 7.0×10⁹ vg/mL+/−15% when cultured in suspension.

In some embodiments, methods further comprise lysis of host cells. In some embodiments, a lower amount of a first vector is required for production of a plurality of rAAV particles, relative to an amount of an Ad5 helper vector required for production of a plurality of rAAV particles.

In some embodiments, rAAV particles have at least one of the following characteristics: (a) improved infectivity, relative to rAAV particles produced with another helper vector, (b) increased expression of at least one payload, relative to rAAV particles produced with another helper vector, or (c) improved transduction, relative to rAAV particles produced with another helper vector. In some embodiments, a plurality of rAAV particles is substantially free of one or both of a helper adenovirus or a herpes virus. In some embodiments, a plurality of rAAV have a reduced level of adenoviral impurities relative to rAAV particles produced with an Ad5 helper vector.

In some embodiments, a first vector lacks a nucleic acid sequence encoding a Fiber protein. In some embodiments, a first vector comprises or consists of a nucleic acid sequence of SEQ ID NO: 1. In some embodiments, a first vector further comprises a nucleic acid sequence of a kanamycin resistance (KanR) gene.

In some embodiments, a third vector further comprises one or both of a sequence encoding a 5′ inverted terminal repeat (ITR) or a sequence encoding a 3′ ITR.

In some embodiments, Ad2 helper polypeptides are oriented in the same direction.

In some embodiments, a higher yield of a plurality of rAAV particles is produced relative to a plurality of rAAV particles produced with a helper vector comprising a nucleic acid sequence of an antibiotic resistance gene other than KanR.

In some embodiments, methods further comprise one or both of isolating or purifying rAAV particles from a host cell.

In another aspect, the disclosure provides compositions comprising a plurality of rAAV particles formed by methods of any aspect or embodiment described herein.

In another aspect, the disclosure provides pharmaceutical compositions comprising a composition of any aspect or embodiment described herein and a pharmaceutically acceptable component.

In another aspect, the disclosure provides methods of delivering a gene therapy to a cell or tissue, comprising: contacting a cell or tissue with a composition (e.g., a pharmaceutical composition) of any aspect or embodiment described herein, thereby delivering a gene therapy to a cell or tissue.

In another aspect, the disclosure provides methods of treating a subject, comprising: administering a composition (e.g., a pharmaceutical composition) of any aspect or embodiment described herein to a subject, thereby treating a subject.

In another aspect, the disclosure provides reaction mixtures comprising: (i) a first vector encoding at least one Ad2 helper polypeptides; (ii) a second vector encoding at least one Rep polypeptide and/or at least one Cap polypeptide; (iii) a third vector encoding a payload; and (iv) a transfection reagent.

In another aspect, the disclosure provides reaction mixtures comprising: (i) a first vector encoding at least one Ad2 helper function polypeptides and at least one Rep polypeptide; (ii) a second vector encoding at least one Cap polypeptide and at least one payload, and (iii) a transfection reagent.

In another aspect, the disclosure provides transfection complexes comprising: (i) a first vector encoding at least one Ad2 helper polypeptides; and (ii) a transfection reagent. In some embodiments, transfection complexes further comprise one or both of: (iii) a second vector encoding at least one Rep polypeptide and/or at least one Cap polypeptide); or (iv) and a third vector encoding at least one payload.

In another aspect, the disclosure provides transfection complexes comprising: (i) a first vector encoding at least one Ad2 helper function polypeptides and at least one Rep polypeptide; (ii) a second vector encoding at least one Cap polypeptide and at least one payload, and (iii) a transfection reagent.

In another aspect, the disclosure provides cultures comprising a plurality of host cells and a reaction mixture of any aspect or embodiment described herein, or a transfection complex of any aspect or embodiment described herein.

In another aspect, the disclosure provides bioreactors comprising a culture of any aspect or embodiment described herein. In some embodiments, a bioreactor comprises at least one of: (i) at least about 1×10³ host cells; (ii) at least about 10 liters of culture media; or (iii) at least about 5 m² of culture media. In some embodiments, a bioreactor is selected from a continuous flow bioreactor, a batch process bioreactor, a perfusion bioreactor, and a fed batch bioreactor. In some embodiments, a bioreactor is held under conditions suitable for formation of a plurality of rAAV particles.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The Figures described below, which together make up the Drawing, are for illustration purposes only, not for limitation.

FIGS. 1A-1D shows an exemplary nucleic acid sequence of a plasmid encoding Adenovirus 2 (Ad2) helper polypeptides (SEQ ID NO: 1; hereinafter “A2 helper plasmid”) derived from the Adenovirus 2 genome (Genbank Accession No. J01917.1). The nucleic acid sequence is 12,010 bp in length.

FIG. 2 shows a plasmid map of the A2 helper plasmid encoding the Ad2 helper polypeptides VA RNA, E4, and E2A oriented from 5′ to the 3′ in direction.

FIGS. 3A-3B shows a comparison of production (AAV titer) of rAAV particles using the A2 helper plasmid relative to two Ad5 helper plasmids designated pAd5-1 and pAd5-2 (each derived from the Adenovirus 5 genome (Genbank Accession No. AY601635)). Transient transfection was performed using adherent HEK293 cells in t-flasks (FIG. 3A) and in the iCELLis® Nano Bioreactor (FIG. 3B).

FIG. 4 shows a comparison of production (AAV titer) of rAAV particles using the A2 helper plasmid relative to Ad5 helper plasmid pAd5-1 with two AAV serotypes and a control average, which is a combination of historic data for pAd5-1 and an AAV serotype. Transient transfection was performed using suspension HEK293 cells in shaker flasks.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

5′ and 3′: The terms “5′” and “3′” are relative terms to define a spatial relationship or directionality between two or more segment of a nucleic acid sequence. Thus, 3′ of a nucleic acid indicates a segment of the nucleic acid that is downstream of another segment, while 5′ indicates a segment of the nucleic acid that is upstream of another segment. For example, 3′ may indicate that a segment is in the 3′ half of the nucleic acid sequence or even at the 3′ end of the nucleic acid sequence. Similarly, 5′ may indicate that a segment is in the 3′ half of the nucleic acid sequence or even at the 5′ end of the nucleic acid sequence. Unless indicated otherwise, the directionality of a nucleic acid will be in the 5′ to 3′ direction of translation.

About or approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Adeno-associated virus (AAT): As used herein, the terms “Adeno-associated virus” and “AAV” refer to viral particles, in whole or in part, of the family Parvoviridae and the genus Dependoparvovirus. AAV is a small replication-defective, nonenveloped virus. AAV includes, but is not limited to, AAV serotype 1, AAV serotype 2, AAV serotype 3 (including serotypes 3A and 3B), AAV serotypes 4, AAV serotypes 5, AAV serotypes 6, AAV serotypes 7, AAV serotypes 8, AAV serotypes 9, AAV serotypes 10, AAV serotypes 11, AAV serotypes 12, AAV serotype 13, snake AAV, avian AAV, bovine AAV, canine AAV, equine AAV, ovine AAV, goat AAV, shrimp AAV, and any variant of any of the foregoing. Wild-type AAV is replication deficient and requires co-infection of cells by a helper virus, e.g., adenovirus, herpes, or vaccinia virus, e.g., an Ad2 or Ad5 virus, or supplementation of helper viral genes, in order to replicate.

Ad2 helper: As used herein, the term “Ad2 helper” refers to the Adenovirus serotype 2 (Ad2) helper virus (e.g., wildtype or recombinantly engineered Ad2 helper virus) and various Ad2 helper genes and/or Ad2 helper polypeptides, including, but not limited, to E1a, E1b, E2a, E4Orf6, VA RNA, and any variant or fragment of any of the foregoing. In some embodiments, an Ad2 helper vector (e.g., plasmid) encodes Ad2 helper polypeptides (e.g., one, two, three, or four of E1 (e.g., E1a and/or E1b), E2A, E4, or VA RNA) necessary to generate functional rAAV particles. In certain embodiments, the Ad2 helper vector is transfected into an E1 complementing cell line (e.g., HEK293). The nucleotide sequence of an Ad2 helper vector and Ad2 helper virus genes can be derived from the Adenovirus 2 genome (Genbank Accession No. J01917.1).

Ad5 helper: As used herein, the term “Ad5 helper” refers to the Adenovirus serotype 5 (Ad5) helper virus (e.g., wildtype or recombinantly engineered Ad5 helper virus) and various Ad5 helper genes and/or Ad5 helper polypeptides, including, but not limited, to E1a, E1b, E2a, E4Orf6, and/or VA RNA. In some embodiments, an Ad5 helper vector (e.g., plasmid) comprises Ad5 helper genes (e.g., one, two, three, or four of E1 (e.g., E1a and/or E1b), E2A, E4, or VA RNA) necessary to generation functional rAAV particles. In certain embodiments, the Ad5 helper vector is transfected into an E1 complementing cell line (e.g., HEK293). The nucleotide sequence of an Ad5 helper vector and Ad5 helper genes can be derived from the Adenovirus 5 genome (Genbank Accession No. AY601635).

Administration: As used herein, the term “administration” refers to the administration of a composition comprising rAAV particles as described herein to a subject. Administration may be by any appropriate route. For example, in some embodiments, administration may be local or systemic administration (e.g., to a mammal, e.g., to a human, e.g., a patient). A composition of the disclosure may be administered by injection or infusion by any route. For example, a composition may be administered by retinal, subretinal, intravitreal, suprachoroidal, intraspinal, intracisterna magna, or intrathecal injection or infusion. Additional exemplary routes of administration may include, but are not limited to, bronchial (e.g., bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (e.g., intratracheal instillation), transdermal, vaginal, and vitreal.

Bioreactor: The term “bioreactor,” as used herein, refers to any vessel used for the growth of a cell culture (e.g., a mammalian cell culture). The bioreactor can be of any size and/or any shape so long as it is useful for culturing a cell culture (e.g., a mammalian cell culture).

Cap polypeptide: As used herein, the term “Cap polypeptide” refers to the structural proteins that form a functional AAV capsid, which can in turn package DNA and infect a target cell. In some embodiments, Cap polypeptides will comprise all of the AAV capsid subunits, but less than all of the capsid subunits may be present as long as a functional capsid is produced. In some embodiments, the nucleic acid sequence encoding Cap polypeptides will be present on a single vector (e.g., plasmid). In some embodiments, the Cap polypeptide comprises an AAV1, AAV2, AAV3, AAV4, AAV5, AAV6, AAV7, AAV8, AAV9, AAV10, or AAV11 Cap polypeptide, or a variant of any of the foregoing. AAV capsid genes and proteins have been described in, e.g., Knipe et al., FIELDS VIROLOGY, Volume 1, (6th ed., Lippincott-Raven Publishers), which is hereby incorporated by reference in its entirety.

Cell Density: As used herein, the term “cell density” refers to that number of cells present in a given volume of medium or the number of cells present in a given surface area. For example, cell density may be represented as viable cells (vc)/cm² of culture medium.

Culture: As used herein, the terms “culture” and “cell culture” refer to a cell population (e.g., a eukaryotic cell population) that is suspended in or covered by a medium under conditions suitable to survival and/or growth of the cell population. As will be clear to those of ordinary skill in the art, these terms can also refer to the combination comprising the cell population and the medium.

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Gene: As used herein, the term “gene” refers to a DNA sequence that codes for a product (e.g., an RNA product and/or a polypeptide product). In some embodiments, a gene includes coding sequence (i.e., a sequence that encodes a particular product). In some embodiments, a gene includes non-coding sequence. In some particular embodiments, a gene may include both coding (e.g., exonic) and non-coding (e.g., intronic) sequence. In some embodiments, a gene may include one or more regulatory elements that, for example, may control or effect one or more aspects of gene expression (e.g., inducible expression, etc.).

Gene therapy: As used herein, the term “gene therapy” refers to insertion or deletion of specific genomic DNA sequences to treat or prevent a disorder or condition for which such therapy is sought. In some embodiments, the insertion or deletion of genomic DNA sequences occurs in specific cells (e.g., target cells). Target cells may be from a mammal and/or may be cells in a mammalian subject. Mammals include but are not limited to humans, dogs, cats, cows, sheep, pigs, llamas, etc. In some embodiments, heterologous DNA is transferred to target cells. The heterologous DNA may be introduced into the selected target cells in a manner such that the heterologous DNA is expressed and a therapeutic product encoded thereby is produced. Additionally or alternatively, the heterologous DNA may in some manner mediate expression of DNA that encodes the therapeutic product, or it may encode a product, such as a peptide or RNA that in some manner mediates or modulates, directly or indirectly, expression of a therapeutic product. Genetic therapy may also be used to deliver nucleic acid encoding a gene product that replaces a defective gene or supplements a gene product produced by the mammal or the cell in which it is introduced. The heterologous DNA encoding the therapeutic product may be modified prior to introduction into the cells of the afflicted host in order to enhance or otherwise alter the product or expression thereof. Genetic therapy may also involve delivery of an inhibitor or repressor or other modulator of gene expression. Such an inhibitor or repressor or other modulator can be a polypeptide, peptide, or nucleic acid (e.g., DNA or RNA). Gene therapy may include in vivo or ex vivo techniques. In some embodiments, viral and non-viral based gene transfer methods can be used to introduce a nucleic acid encoding a polypeptide of interest or to introduce a therapeutic nucleic acid into mammalian cells or target tissues. Non-viral vector delivery systems include DNA plasmids, naked nucleic acid, and nucleic acid complexed with a delivery vehicle, such as poloxamers or liposomes. Viral vector delivery systems include DNA and RNA viruses, which have either episomal or integrated genomes after delivery to the cell. For a review of gene therapy procedures, see Anderson, Science 256:808-813 (1992); Miller, Nature 357:455-460 (1992); Feuerbach et al., Kidney International 49:1791-1794 (1996); Urnov et al., Nature Reviews Genetics 11, 636-646 (2010); and Collins et al., Proceedings Biologicial Sciences/The Royal Society, 282(1821):pii 20143003 (2015), each of which is hereby incorporated by reference in its entirety.

Host Cell: As used herein, the term “host cell” refers a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence).

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve,” “increase,” “inhibit,” or “reduce”: As used herein the terms “improve”, “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single sample, e.g., of a culture medium) under otherwise comparable conditions absent presence of (e.g., prior to and/or after) a particular agent or treatment, or in presence of an appropriate comparable reference agent. In some embodiments, an appropriate reference measurement may be or comprise a measurement in a comparable system known or expected to respond in a particular way, in presence of the relevant agent or treatment.

Medium: As used herein, the terms “medium,” “culture medium,” and “growth medium” refer to a solution comprising nutrients to nourish cells (e.g., growing cells, e.g., eukaryotic cells). Typically, these solutions provide essential and non-essential amino acids, vitamins, energy sources, lipids, and trace elements required by the cell for survival and/or minimal growth. The solution can also comprise components that enhance survival and/or growth above the minimal rate, including hormones and growth factors. The solution can be formulated to a pH and concentration of one or more salts that are optimal for cellular survival and/or proliferation. For example, the medium can also be a “defined medium” or “chemically defined medium,” e.g., a serum-free medium that contains no proteins, hydrolysates, or components of unknown composition. Defined media are free of animal-derived components and all components have a known chemical structure. One of skill in the art understands a defined medium can comprise recombinant polypeptides, for example, but not limited to, hormones, cytokines, interleukins, and/or other signaling molecules.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly- refers to a nucleic acid containing 2 to about 10,000, 2 to about 50,000, or 2 to about 100,000 nucleotide monomer units. In some embodiments, the prefix oligo- refers to a nucleic acid containing 2 to about 200 nucleotide monomer units. In accordance with the methods and compositions described herein, in some embodiments, an RNA comprises a short hairpin RNA (shRNA), small interfering RNA (siRNA), mRNA, snRNA, CRISPR/Cas guide RNA, microRNA (miRNA), and/or a precursor thereof.

Payload: As used herein, the term “payload” refers to a nucleic acid sequence of interest (e.g., comprising a sequence that encodes a target payload, such as a target polypeptide) that is desired to be introduced into a cell, tissue, organ, organism, and/or system comprising cells. A target payload can be a heterologous protein with a therapeutic purpose, e.g., an enzyme or antibody. The target payload can be a heterologous nucleic acid with a therapeutic purpose, e.g., an miRNA, siRNA, shRNA, mRNA, snRNA, or CRISPR/Cas guide RNA, or a precursor thereof. One of skill in the art will recognize that the target payload can be selected from any heterologous protein or nucleic acid of interest. As used herein, “encode” or “encodes” means directs the expression of or processed into. For example, as used herein, a nucleic acid encodes a polypeptide sequence if it directs the expression of that polypeptide sequence. As another example, as used herein, a nucleic acid precursor (e.g., a pri-miRNA or pre-miRNA) encodes a further processed version of the nucleic acid (e.g., mature miRNA) if it is processed into the further processed version.

Pharmaceutical composition: As used herein, the term “pharmaceutical composition” refers to a composition comprising rAAV particles that is suitable for administration to a human or animal subject. In some embodiments, a pharmaceutical composition comprises an active agent formulated together with one or more pharmaceutically acceptable carriers. In some embodiments, the active agent is present in a unit dose amount appropriate for administration in a therapeutic regimen. In some embodiments, a therapeutic regimen comprises one or more doses administered according to a schedule that has been determined to achieve a desired therapeutic effect when administered to a subject or population in need thereof (e.g., by a statistically significant probability). A pharmaceutical composition may be specially formulated for administration in solid or liquid form. In some embodiments, a pharmaceutical composition is formulated for administration by parenteral administration, such as by subcutaneous, intramuscular, intravenous or epidural injection. In some embodiments, a pharmaceutical composition is formulated as a sterile solution or suspension, e.g., in a sustained-release formulation. Pharmaceutical compositions of the disclosure may be formulated for administration by injection or infusion (e.g., subcutaneous, intramuscular, intravenous or epidural injection or infusion). For example, compositions may be formulated for administration by retinal, subretinal, intravitreal, suprachoroidal, intraspinal, intracisterna magna, or intrathecal injection or infusion. In some embodiments, a pharmaceutical composition is intended and suitable for administration to a human subject. In some embodiments, a pharmaceutical composition is substantially free of contaminants (e.g., sterile and substantially pyrogen-free). Formulations of the pharmaceutical compositions may include, but are not limited to, formulations for oral administration, such as drenches (aqueous or non-aqueous solutions or suspensions), tablets (e.g., targeted for buccal, sublingual, and systemic absorption), boluses, powders, granules, pastes for application to the tongue; topical application, such as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (e.g., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc) that is transgenic for or otherwise has been manipulated to express a gene or genes, or gene components that encode and/or direct expression of the polypeptide or one or more component(s), portion(s), element(s), or domain(s) thereof and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Recombinant AAV (rAAV) particle: A “recombinant AAV particle”, or “rAAV particle,” as used herein, refers to an infectious, replication-defective viral particle comprising an AAV protein shell encapsulating a payload that is flanked on both sides by ITRs. An AAV particle is produced in a suitable host cell (e.g., a HEK293 cell). For example, the host cell is transfected with at least one vector encoding one or more helper polypeptides (e.g., Ad2 helper polypeptides), at least one Rep polypeptide, at least one Cap polypeptide, and at least one payload (e.g., for polypeptide expression or a therapeutic nucleic acid), such that the host cell is capable of producing the Rep and Cap polypeptides necessary for packing the rAAV particle. rAAV particles may be used for subsequent gene delivery.

Rep polypeptide: The term “Rep polypeptide”, as used herein, refers to the AAV non-structural proteins that mediate AAV replication for the production of AAV particles. The AAV replication genes and proteins have been described in, e.g., Knipe et al., FIELDS VIROLOGY, Volume 1, (6th ed., Lippincott-Raven Publishers), which is hereby incorporated by reference in its entirety.

Seeding: The term “seeding” as used herein refers to the process of providing a cell culture to a vessel (e.g., a bioreactor or culture flask). For example, the process of providing a cell culture may include propagation of the cells in another bioreactor or vessel before providing to the bioreactor or other vessel. The cells have been frozen and thawed immediately prior to providing them to the bioreactor or vessel. The term “seeding” refers to providing any number of cells, including a single cell.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein, e.g., a neurological disease or disorder or a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Titer: As used herein, the term “titer” refers to the quantity of virus in a given volume. Titer, for example, can be expressed as viral genome copies (vg) per given volume or plaque forming units (pfu) per given volume. In some embodiments, titer can be expressed as number of capsids per given volume.

Transfection: As used herein, the term “transfection” refers to the introduction of nucleic acid molecules, such as DNA or RNA (e.g. mRNA) molecules, into cells, such as eukaryotic cells (e.g., mammalian cells). For example, transfection can include vector-based transfection, viral-based transfection, electroporation, lipofection (e.g., with cationic lipids and/or liposomes), calcium phosphate precipitation, nanoparticle-based transfection, and/or transfection based on cationic polymers (e.g., DEAE-dextran or polyethylenimine).

Treating: As used herein, the term “treating” refers to providing treatment, e.g., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a condition, disease, or disorder, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition comprising rAAV particles of the disclosure can be administered to a subject who has developed a disorder or is at increased risk of developing such a disorder relative to a member of the general population. A composition of the disclosure can be administered prophylactically or before development of any symptom or manifestation of the condition. Typically, in this case, the subject will be at risk of developing the condition.

Vector: As used herein, the term “vector” refers to a molecule comprising a nucleic acid molecule, where the vector is capable of transporting the nucleic acid molecule into a cell. By way of non-limiting example, one type of vector is a “plasmid,” which refers to a circular double stranded DNA loop into which additional DNA segments may be ligated. Another type of vector is a viral vector, wherein additional DNA segments may be packaged into a viral capsid and can be transferred into another cell and/or organism. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference in its entirety.

### DETAILED DESCRIPTION OF CERTAIN EMBODIMENTS

The present disclosure provides, inter alia, improved compositions and methods for the production of recombinant AAVs (rAAVs). The present disclosure is based, in part, on the discovery of a vector (e.g., a plasmid) encoding one or more Adenovirus 2 (Ad2) helper polypeptides that surprisingly results in improved rAAV production, such as in a large-scale preparation of rAAV particles. Thus, the present disclosure provides, inter alia, a scalable rAAV manufacturing process using an Ad2 helper vector that efficiently generates high titer, high purity, and/or potent quantities of rAAV particles.

Without wishing to be bound by theory, it is believed that, in some embodiments, the use of an Ad2 helper vector described herein leads to production of rAAV particles having improved characteristics and/or leads to more scalable manufacturing methods for rAAV particles, relative to a reference vector. In certain embodiments, the reference vector is an Ad5 helper vector, e.g., an Ad5 helper vector described herein. In some instances, as compared to a reference vector, the AAV particles are produced at a higher titer when the Ad2 helper vector is used. In some instances, as compared to a reference vector (e.g. an Ad5 helper vector described herein), there is improved productivity (e.g., titer) of AAV particles, e.g., improved productivity by at least about 10%, 15%, 20%, 25%, 26%, 27%, 28%, 29%, 30%, 35%, 40%, 45%, 50%, 55%, 60%, 65%, 70%, 75%, 80%, 85%, 90%, or more. In some instances, as compared to a reference vector (e.g. an Ad5 helper vector described herein), there is improved productivity (e.g., titer) of AAV particles, e.g., improved productivity by at least about 10% to about 60%, e.g., about 20% to about 50%.

In some instances, as compared with a reference vector, a lower amount of the Ad2 helper vector is required for production of the plurality of rAAV particles. In some instances, as compared with a reference vector, the rAAV particles produced using the Ad2 helper vector have improved infectivity. In some instances, as compared with a reference vector, the rAAV particles produced using the Ad2 helper vector have increased payload expression or amount. In some instances, as compared with a reference vector, the rAAV particles produced using the Ad2 helper vector exhibit improved transduction. In some instances, as compared with a reference vector, the rAAV particles produced using the Ad2 helper vector exhibit greater purity (e.g., reduced adenoviral contamination). In some embodiments, no Fiber coding sequence is included in rAAV generation methods—this is believed to lead to reduced adenoviral impurities in the rAAV particles. In some embodiments, less overall adenoviral genome sequence is included in the rAAV generation methods—this is also believed to lead to reduced adenoviral impurities in the rAAV particles. In some embodiments, less impurities, e.g., adenoviral impurities in the rAAV particles produced by the methods described herein confers a safety benefit when the rAAV-containing compositions are used for gene therapy. In some instances, as compared with a reference vector, the rAAV particles produced using the Ad2 are on a larger production scale. In some instances, as compared with a reference vector, there is improved formation of transfection complexes using the Ad2 helper vector. In some instances, as compared with a reference vector, the yield of Ad2 helper vector (e.g., total amount DNA) produced in host cells (e.g., bacterial cells, e.g., E. coli cells) is greater. Thus, the present disclosure represents a significant advancement in production of rAAV particles, e.g., that are useful in compositions for gene therapy and treating diseases and disorders with gene therapy methods.

The Ad2 helper plasmid has advantages over other helper plasmids, such as Ad5 helper plasmids containing components derived from the Adenovirus 5 genome (Genbank Accession No. AY601635). Exemplary Ad5 helper plasmids include pAF6 (e.g., Plasmid #112867 at Addgene; or as described in Dudek et al. Mol. Therapy 28.2(2020):367-8 or in WO2018/160585 or in Lock et al. Human Gene Therapy 21(2010):1259-71) and pAldX-80 (Aldevron), both of which are derived from the Adenovirus 5 genome (Genbank Accession No. AY601635). The Ad2 helper plasmid contains less overall Adenoviral genome sequence. For example, the nucleic acid sequence of the Ad2 helper plasmid contains only 9267 nucleotides of Adenoviral genome, whereas Ad5 helper plasmids contain a greater number of nucleotides of Adenoviral genome, e.g., greater than 9500, 10000, 10500, 11000, 11500, 12000, or more nucleotides. For example, the pAF6 and pALD-X80 helper plasmids contain 12397 and 12182 nucleotides of Adenoviral genome, respectively. A reduction in plasmid size provides a reduction in transfection material requirements and confers a benefit to plasmid yield during manufacturing. Notably, Ad2 helper plasmid does not contain the coding region for Fiber protein (Genbank Accession No. AP 000226.1), which reduces the amount of adenoviral impurities and overall plasmid size by 1745 nucleotides.

Vectors

Many forms of vectors can be used in methods of producing rAAV particles described herein. Non-limiting examples of vectors include plasmids, bacteriophage vectors, cosmids, phagemids, artificial chromosomes, and viral vectors (e.g., vectors suitable for gene therapy). A vector genetic element may be delivered by any suitable method known in the art, e.g., to those with skill in nucleic acid manipulation and include genetic engineering, recombinant engineering, and synthetic techniques (See, e.g., Sambrook et al, Molecular Cloning: A Laboratory Manual, Cold Spring Harbor Press, Cold Spring Harbor, N.Y.).

In some embodiments, a vector encodes at least one helper polypeptide (e.g., Ad2 helper polypeptide) or a fragment thereof. In some embodiments, a vector encodes at least one Rep polypeptide and/or at least one Cap polypeptide. In some embodiments, a vector encodes at least one payload (e.g., for expression of polypeptide or as an inhibitory nucleic acid). In some embodiments, a vector encodes at least one Ad2 helper function polypeptide and at least one Rep polypeptide. In some embodiments, a vector encodes at least one Cap polypeptide and at least one payload.

A vector can include conventional control elements operably linked to a nucleic acid encoding any polypeptide or payload described herein, in a manner that permits transcription, translation and/or expression in a cell transfected with a vector described herein. Expression control sequences include appropriate transcription initiation, termination, promoter and enhancer sequences; efficient RNA processing signals, such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters that are native, constitutive, inducible, and/or tissue-specific, are known in the art and may be included in a vector described herein.

Examples of constitutive promoters include, but are not limited to, a retroviral Rous sarcoma virus (RSV) LTR promoter (optionally with the RSV enhancer), a cytomegalovirus (CMV) promoter (optionally with CMV enhancer), an SV40 promoter, and an dihydrofolate reductase promoter.

Inducible promoters allow regulation of gene expression and can be regulated by exogenously supplied compounds, environmental factors, such as temperature, or the presence of a specific physiological state (e.g., acute phase, a particular differentiation state of the cell, or in replicating cells only). Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include a zinc-inducible sheep metallothionine (MT) promoter, a dexamethasone (Dex)-inducible mouse mammary tumor virus (MMTV) promoter, a T7 polymerase promoter system, an ecdysone insect promoter, a tetracycline-repressible system, a tetracycline-inducible system, a RU486-inducible system, and an rapamycin-inducible system. Still other types of inducible promoters that may be useful are regulated by a specific physiological state, such as temperature, acute phase, a particular differentiation state of the cell, or in replicating cells only.

In another embodiment, a native promoter or fragment thereof for a nucleic acid encoding any polypeptide or payload described herein may be used. In some embodiments, other native expression control elements, such as enhancer elements, polyadenylation sites, or Kozak consensus sequences, may also be used to mimic native expression.

Vector Encoding Helper Polypeptides

The present disclosure, among other things, provides vectors (e.g., plasmids) encoding at least one helper polypeptide. AAV is a helper-dependent DNA parvovirus, which belongs to the genus Dependovirus. Production of recombination AAV requires co-infection with a related virus (e.g., adenovirus, herpes, or vaccinia virus) or a helper vector encoding helper polypeptides, such as structural proteins and proteins for viral genome replication.

A helper vector (e.g., an Ad2 helper vector) can comprise nucleotide sequences for non-AAV derived viral and/or cellular functions upon which AAV is dependent for replication, which may include, but are not limited to, activation of gene transcription, stage specific mRNA splicing, DNA replication, synthesis of at least one Cap polypeptide, and/or capsid assembly. Viral-based helper polypeptides can be derived from any known helper viruses such as adenovirus, herpesvirus, vaccinia virus, or a combination thereof. Thus, a helper vector (e.g., a plasmid) for culturing of the host cell can comprise sufficient helper function to permit packaging of the recombinant AAV vector into the AAV capsid polypeptides.

In some embodiments, a helper vector comprises an Ad2 helper vector. In certain embodiments, a nucleic acid sequence of an Ad2 helper vector is derived from an Adenovirus 2 genome (Genbank Accession No. J01917.1). In some embodiments, a helper vector comprises an Ad5 helper vector. In certain embodiments, a nucleic acid sequence of an Ad5 helper vector is derived from an Adenovirus 5 genome (Genbank Accession No. AY601635).

Helper polypeptides (e.g., Ad2 helper polypeptides) can comprise at least one, two, three, or four of E1, E2A, E4, or VA RNA. In some embodiments, E1 comprises E1a and/or E1b. In some embodiments, one or both of E2A and VA RNA increase stability and/or efficiency of AAV mRNA translation, such as for cap gene transcripts. In some embodiments, E4 facilitates DNA replication. In some embodiments, E1a comprises a transactivator (e.g., regulating activity of at least one Ad gene, AAV rep gene, and/or AAV cap gene). In some embodiments, E1b comprises a viral mRNA transport. Helper polypeptides are described in further detail in Coura and Nardi, A role for adeno-associated viral vectors in gene therapy, Genetics and Molecular Biology, 31(1): 1-11 (2008), which is hereby incorporated by reference in its entirety.

In some embodiments, a helper vector (e.g., an Ad2 helper vector) comprises a selection marker. Exemplary selection markers include, but are not limited to, antibiotic resistance genes. In some embodiments, an antibiotic resistance gene is not a gene encoding penicillin. In some embodiments, an antibiotic resistance gene is not a gene encoding a penicillin-derivative. In some embodiments, an antibiotic resistance gene comprises an antibiotic resistance gene chosen from kanamycin, puromycin, neomycin, hygromycin, blasticidin, gentamycin, Gr18, or zeocin. In certain embodiments, an antibiotic resistance gene comprises an antibiotic resistance gene for kanamycin.

In some embodiments, nucleic acids encoding helper polypeptides (e.g., Ad2 helper polypeptides) are oriented in the same direction (e.g., 5′ to 3′) on a helper vector (e.g., Ad2 helper vector). In some embodiments, nucleic acids encoding helper polypeptides are transcribed in the same direction from a helper vector. In certain embodiments, helper polypeptides comprise VA RNA and E4 oriented in the same direction on a helper vector. In certain embodiments, helper polypeptides comprise E4 and E2A oriented in the same direction on a helper vector. In certain embodiments, helper polypeptides comprise VA RNA, E4, and E2A oriented from 5′ to 3′ in direction on a helper vector. In some embodiments, a helper vector does not comprise a nucleic acid sequence encoding a Fiber protein or a fragment thereof (e.g., does not comprise a nucleic acid sequence of Genbank Accession No. AP_000226.1 or a fragment thereof).

In some embodiments, a helper vector comprises a nucleic acid sequence at least about 90%, at least about 91%, at least about 92%, at least about 93%, at least about 94%, at least about 95%, at least about 96%, at least about 97%, at least about 98%, at least about 99%, or more identical to SEQ ID NO: 1. In certain embodiments, embodiments, a helper vector comprises a nucleic acid sequence of SEQ ID NO: 1. In certain embodiments, a helper vector consists of a nucleic acid sequence of SEQ ID NO: 1. In some embodiments, a helper vector comprises a double-stranded DNA sequence that comprises (i) a first strand comprising a first sequence that is at least about 90%, 91%, 92%, 93%, 94%, 95%, 96%, 97%, 98%, 99%, or 100% identical to SEQ ID NO: 1; and (ii) a second strand comprising a second sequence that is complementary to the first sequence.

Vector Encoding Rep and/or Cap Polypeptides

The present disclosure, among other things, provides vectors (e.g., plasmids) encoding at least one Rep polypeptide and/or at least one Cap polypeptide. Production of rAAV particles can include culturing of a host cell with at least one Rep polypeptide and at least one Cap polypeptide. Rep proteins (e.g., one, two, three, or four Rep78, Rep68, Rep52, and Rep40) are involved in viral DNA replication, resolution of replicative intermediates, and generation of single-stranded genomes. In some embodiments, a vector comprises a nucleic acid sequence encoding one, two, three, or four of Rep78, Rep68, Rep52, or Rep40, or a variant of any of the foregoing.

In some embodiments, a Rep polypeptide comprises a nucleic acid sequence derived from an AAV2 serotype. For example, a nucleic acid sequence encoding a Rep polypeptide may be derived from the AAV2 genome (as found in Accession No. NC_001401). In some embodiments, a Rep polypeptide comprises an AAV2 Rep polypeptide operably linked to a p5 and/or p19 promotor (as found in Accession No. NC_001401). In some embodiments, a Rep polypeptide comprises an amino acid sequence of YP_680422.1 or a fragment thereof. In some embodiments, a promoter is operably linked to a nucleic acid sequence encoding at least one Rep polypeptide. In certain embodiments, a promoter operably linked to a nucleic acid sequence encoding at least one Rep polypeptide comprises a p5 and/or p19 promoter. In some embodiments, a wildtype promoter of AAV2 or a variant thereof is operably linked to a nucleic acid sequence encoding at least one Rep polypeptide. In some embodiments, a promoter (e.g., a p5 promoter) regulating expression of at least one Rep polypeptide is located in a different location on a vector than a wildtype promoter of AAV2 or a variant thereof. In certain embodiments, a promoter (e.g., a p5 promoter) is located 3′ of a nucleic acid encoding at least one Rep polypeptide. In certain embodiments, a promoter (e.g., a p5 promoter) is located 5′ of a nucleic acid encoding at least one Rep polypeptide.

Cap polypeptides (e.g., VP1, VP2, and VP3) are structural proteins comprising a Capsid. In some embodiments, a vector comprises a nucleic acid sequence encoding one, two, or three of VP1, VP2, and VP3. In some embodiments, a vector comprises a nucleic acid sequence encoding at least one Cap polypeptide and at least one Rep polypeptide. In other embodiments, a vector encodes at least one Cap polypeptide and a separate vector encodes at least one Rep polypeptide. In some embodiments, a Cap polypeptide comprises a nucleic acid sequence derived from an AAV1, AAV2, AAV3, AAV4, AAV5, AAV6, AAV7, AAV8, AAV9, AAV10, or AAV11 serotype, or a variant of any of the foregoing, such as Rec2 or Rec3. For example, a nucleic acid sequence encoding a Cap polypeptide may be derived from a known AAV genome sequence including, but not limited to: AAV1 Accession No. NC_002077 or AF063497; AAV2 Accession No. NC_001401; AAV3 Accession No. NC_001729; AAV3B Accession No. NC_001863; AAV4 Accession No. NC_001829; AAV5 Accession No. Y18065 or AF085716; Accession No. AAV6 NC_001862; Avian AAV ATCC VR-865 AY186198, AY629583, or NC_004828; Avian AAV strain DA-1 NC_006263, AY629583; or Bovine AAV NC_005889, AY388617. In certain embodiments, a nucleic acid sequence encoding a Cap polypeptide is derived from an AAV genome sequence or a variant thereof as described in U.S. Pat. No. 7,906,111, which is hereby incorporated by reference in its entirety. In certain embodiments, a nucleic acid sequence encoding a Cap polypeptide is derived from an AAV genome sequence or a variant thereof as described in International Publication No. WO 2018/160582, which is hereby incorporated by reference in its entirety.

In certain embodiments, a Cap polypeptide comprises a nucleic acid sequence derived from an AAV2 serotype, or a variant thereof. In certain embodiments, a Cap polypeptide comprises a nucleic acid sequence derived from an AAV2 serotype, or a variant thereof. In certain embodiments, a Cap polypeptide comprises a nucleic acid sequence derived from an AAV5 serotype, or a variant thereof. In certain embodiments, a Cap polypeptide comprises a nucleic acid sequence derived from an AAV8 serotype, or a variant thereof. In certain embodiments, a Cap polypeptide comprises a nucleic acid sequence derived from an AAV9 serotype, or a variant thereof. In certain embodiments, a Cap polypeptide comprises a nucleic acid sequence derived from AAVhu68 or a variant thereof. In certain embodiments, a Cap polypeptide comprises a nucleic acid sequence derived from AAVrh10 or a variant thereof.

In some embodiments, a promoter is operably linked to a nucleic acid sequence encoding at least one Cap polypeptide. In some embodiments, a wildtype promoter of AAV1, AAV2, AAV3, AAV4, AAV5, AAV6, AAV7, AAV8, AAV9, AAV10, or AAV11, or a variant of any of the foregoing, is operably linked to a nucleic acid sequence encoding at least one Rep polypeptide. In certain embodiments, a p40 promoter is operably linked to a nucleic acid sequence encoding at least one Cap polypeptide.

Vector Encoding Payload

The present disclosure, among other things, provides vectors (e.g., plasmids) encoding at least one payload. A payload sequence is generally a sequence of interest that is desired to be introduced into a cell, tissue, organ, or organism.

In some embodiments, a payload is flanked by inverted terminal repeats (ITRs). The AAV sequences of a rAAV vector typically comprise cis-acting 5′ and 3′ inverted terminal repeat (ITR) sequences (See, e.g., B. J. Carter, in “Handbook of Parvoviruses,” ed., P. Tijsser, CRC Press, pp. 155-168 (1990), which is hereby incorporated by reference in its entirety). ITR sequences are typically about 145 bp in length. In some embodiments, one or both of a 5′ITR or a 3′ ITR nucleic acid sequence are modified relative to a known ITR nucleic acid sequence. Modification of ITR nucleic acid sequences is within one of skill in the art (See, e.g., Sambrook et al, “Molecular Cloning. A Laboratory Manual”, 2d ed., Cold Spring Harbor Laboratory, New York (1989); and K. Fisher et al., J Virol., 70:520 532 (1996), each of which is hereby incorporated by reference in its entirety). AAV ITR sequences may be obtained from any known AAV, including mammalian AAV types.

In some embodiments, a payload is a heterologous protein with a therapeutic purpose, e.g., an enzyme, cytokine, antibody, receptor, fusion protein, or chimeric polypeptide. In some embodiments, a payload is linked to a secretion signal sequence for secretion of an expressed polypeptide from a host cell. In some embodiments, a payload is a heterologous nucleic acid with a therapeutic purpose, e.g., an miRNA, siRNA, shRNA, mRNA, snRNA, or CRISPR/Cas guide RNA, or a precursor thereof. One of skill in the art will recognize that a payload can be selected from any heterologous protein or nucleic acid of interest. In some embodiments, a payload sequence comprises one or more aptamer-binding domains or polypeptide-binding domains (e.g., transcription factor binding domains). A vector will also typically include other regulatory elements (e.g., promoters, introns, and/or enhancers) to regulate expression or amount of a payload in a cell or tissue.

In accordance with various embodiments, a payload sequence can be of any length, e.g., between 2 and 10,000 nucleotides in length or any integer value there between. In some embodiments, a nucleic acid sequence encoding a payload comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides. In some embodiments, a nucleic acid sequence encoding a payload comprises between 50 and 25,000 nucleotides in length, between 100 and 20,000 nucleotides in length, between 500 and 10,000 nucleotides in length, between 1,000 and 8,000 nucleotides in length, and/or between 2,000 and 5,000 nucleotides in length.

Host Cells

The present disclosure, among other things, provides host cells for transfection with at least one vector as described herein for production of rAAV particles. A host cell includes a progeny cell of an original cell transfected with at least one vector described herein. A progeny cell of a parental cell may not be substantially identical in morphology or genomic content as a parent cell due to natural, accidental, or deliberate mutation.

Components for a host cell to produce rAAV particles may be provided in trans on at least one vector. A stable host cell may comprise at least one polypeptide to produce rAAV particles using methods known to those of skill in the art. In some embodiments, a stable host cell comprises at least one polypeptide under control of an inducible promoter. In other embodiments, a stable host cell comprises at least one polypeptide under control of a constitutive promoter. For example, a stable host cell (e.g., a HEK293 cell) may comprise a nucleic acid encoding an E1 helper polypeptide under the control of a constitutive promoter. Other stable host cells may be generated by one of skill in the art using routine methods.

Exemplary host cells include prokaryotes or eukaryotes (single-cell or multiple-cell), bacterial cells (e.g., strains of E. coli, Bacillus spp., Streptomyces spp), mycobacteria cells, fungal cells, yeast cells (e.g., S. cerevisiae, S. pombe, P. pastoris, P. methanolica), plant cells, insect cells (e.g., SF-9, SF-21, baculovirus-infected insect cells, or Trichoplusia ni), non-human animal cells, human cells, or cell fusions, such as hybridomas or quadromas. In some embodiments the host cell is a mammalian cell. In some embodiments, the host cell is a human, monkey, ape, hamster, rat, or mouse cell.

In some embodiments, the host cell is selected from a kidney cell (e.g., HEK293, 293 EBNA, MSR 293, MDCK, HaK, or BHK), CHO cell (e.g., CHO K1, DXB-1 1 CHO, or Veggie-CHO), HeLa cell, COS cell (e.g., COS-7), retinal cell, Vero cell, CV1 cell, HepG2 cell, WI38 cell, MRC 5 cell, Colo205 cell, HB 8065 cell, HL-60 cell (e.g., BHK21), Jurkat cell, Daudi cell, A431 cell (epidermal), CV-1 cell, U937 cell, 3T3 cell, L cell, C127 cell, SP2/0 cell, NS-0 cell, MMT 060562 cell, Sertoli cell, BRL 3 A cell, HT1080 cell, myeloma cell, tumor cell, or a cell line derived from an aforementioned cell.

In some embodiments, the host cell comprises a kidney cell (e.g., HEK293, 293 EBNA, MSR 293, MDCK, HaK, or BHK). In certain embodiments, the host cell comprises a HEK293 cell. In some embodiments, the host cell (e.g., a HEK 293 cell) comprises or expresses an E1 polypeptide. In some embodiments, the host cell does not comprise or express an E1 polypeptide. In some embodiments, the host cell comprises a CHO cell (e.g., CHO-K, DXB-1 1 CHO, or Veggie-CHO). In certain embodiments, the host cell comprises a CHO-K cell. In certain embodiments, the host cell comprises a HeLa cell.

In some embodiments, host cells are or comprise adherent cells. In some embodiments, at least 10%+/−15%, at least 15+/−15%, at least 20+/−15%, at least 25+/−15%, at least 30+/−15%, at least 35+/−15%, at least 40+/−15%, at least 45+/−15%, at least 50+/−15%, at least 55+/−15%, at least 60+/−15%, at least 65+/−15%, at least 70+/−15%, at least 75+/−15%, at least 80+/−15%, at least 85+/−15%, at least 90+/−15%, at least 95+/−15%, at least 99+/−15%, or more host cells in a culture are adherent.

In some embodiments, host cells are or comprise suspension cells. In some embodiments, at least 10%+/−15%, at least 15+/−15%, at least 20+/−15%, at least 25+/−15%, at least 30+/−15%, at least 35+/−15%, at least 40+/−15%, at least 45+/−15%, at least 50+/−15%, at least 55+/−15%, at least 60+/−15%, at least 65+/−15%, at least 70+/−15%, at least 75+/−15%, at least 80+/−15%, at least 85+/−15%, at least 90+/−15%, at least 95+/−15%, at least 99+/−15%, or more host cells in culture are suspended.

In some embodiments, prior to transfection, host cells (e.g., adherent or suspended host cells) are seeded at a certain density. In some embodiments, prior to transfection, host cells (e.g., adherent host cells) are seeded at a density of at least about 1.0×10⁴ viable cells (vc)/cm², e.g., at a density of about 1.0×10⁴ vc/cm² to about 2.0×10⁴ vc/cm², e.g., about 1.0×10⁴ vc/cm², about 1.1×10⁴ vc/cm², about 1.2×10⁴ vc/cm², about 1.3×10⁴ vc/cm², about 1.4×10⁴ vc/cm², about 1.5×10⁴ vc/cm², about 1.6×10⁴ vc/cm², about 1.7×10⁴ vc/cm², about 1.8×10⁴ vc/cm², about 1.9×10⁴ vc/cm², or about 2.0×10⁴ vc/cm². In some embodiments, prior to transfection, host cells (e.g., suspended host cells) are seeded at a density of at least 1.0×10⁶ vc/cm²+/−15%, e.g., at a density of 1.0×10⁶ vc/cm²+/−15% to 2.0×10⁶ vc/cm²+/−15%, e.g., 1.0×10⁶ vc/cm²+/−15%, 1.1×10⁶ vc/cm²+/−15%, 1.2×10⁶ vc/cm²+/−15%, 1.3×10⁶ vc/cm²+/−15%, 1.4×10⁶ vc/cm²+/−15%, 1.5×10⁶ vc/cm²+/−15%, 1.6×10⁶ vc/cm²+/−15%, 1.7×10⁶ vc/cm²+/−15%, 1.8×10⁶ vc/cm²+/−15%, 1.9×10⁶ vc/cm²+/−15%, or 2.0×10⁶ vc/cm²+/−15%.

Transfection

The present disclosure, among other things, provides methods for transfection and/or transduction of a host cell with at least one vector described herein for production of rAAV particles. A host cell (e.g., a mammalian host cell, e.g., a HEK293) can be transfected with: at least one helper polypeptide (e.g., at least one Ad2 helper polypeptides), at least one Rep polypeptide or a fragment thereof, at least one Cap polypeptide or a fragment thereof, and at least one payload (e.g., for polypeptide expression or an inhibitory nucleic acid). In some embodiments, transfection comprises transient transfection. In some embodiments, the disclosure provides transfected host cells comprising two or three vectors as described herein.

In some embodiments, the method comprises transfecting a host cell with three vectors. In some embodiments, the three vectors comprise: (i) a first vector encoding at least one helper polypeptide (e.g., Ad2 helper polypeptide) or a fragment thereof; (ii) a second vector encoding at least one Rep polypeptide or a fragment thereof and at least one Cap polypeptide or a fragment thereof; and (iii) a third vector encoding at least one payload.

In some embodiments, the method comprises transfecting a host cell with two vectors. In some embodiments, the two vectors comprises (i) a first vector encoding at least one Ad2 helper function polypeptide and at least one Rep polypeptide; and (ii) a second vector encoding at least one Cap polypeptide and at least one payload.

In some embodiments, a lower amount of an Ad2 helper vector and/or total vectors is required for transfection to produce a plurality of rAAV particles, e.g., relative to an amount of a reference helper vector (e.g., Ad5 helper vector) and/or reference total vectors required for transfection to produce a plurality of rAAV particles under substantially similar conditions in host cells (e.g., HEK293 cells), e.g., a lower amount of an Ad2 helper vector and/or total vectors by at least about 1%, 2%, 3%, 4%, 5%, 10%, 15%, 20%, 25%, 30%, 40%, 50%, 55%, 60%, 65%, 70%, 75%, 80%, or more, relative to an amount of a reference helper vector and/or reference total vectors. In some embodiments, equal to or less than about 1 ug DNA of Ad2 helper vector and/or total vectors per 1.0×10⁶ vc/cm²+/−15% is required for transfection to produce a plurality of rAAV particles, e.g., equal to or less than about 0.9 ug DNA per 1.0×10⁶ vc/cm²+/−15%, about 0.8 ug DNA per 1.0×10⁶ vc/cm²+/−15%, about 0.7 ug DNA per 1.0×10⁶ vc/cm²+/−15%, about 0.6 ug DNA per 1.0×10⁶ vc/cm²+/−15%, about 0.5 ug DNA per 1.0×10⁶ vc/cm²+/−15%, about 0.4 ug DNA per 1.0×10⁶ vc/cm²+/−15%, about 0.3 ug DNA per 1.0×10⁶ vc/cm²+/−15%, about 0.2 ug DNA per 1.0×10⁶ vc/cm²+/−15%, or about 0.1 ug DNA per 1.0×10⁶ vc/cm²+/−15% of Ad2 helper vector and/or total vectors is required for transfection to produce a plurality of rAAV particles (e.g., in adherent culture). In some embodiments, equal to or less than about 4 ug/ml DNA of Ad2 helper vector and/or total vectors is required for transfection to produce a plurality of rAAV particles, e.g., equal to or less than about 3.5 ug/ml, about 3.0 ug/ml, about 2.5 ug/ml, about 2.0 ug/ml, about 1.5 ug/ml, about 1.0 ug/ml, or about 0.5 ug/ml DNA of Ad2 helper vector and/or total vectors is required for transfection to produce a plurality of rAAV particles (e.g., in suspension culture).

In some embodiments, following transfection, yield of an Ad2 helper vector in host cells (e.g., bacterial cells, e.g., E. coli cells) is greater than yield of a reference helper vector (e.g., Ad5 helper vector) transfected under substantially similar conditions in host cells (e.g., bacterial cells, e.g., E. coli cells).

In accordance with various embodiments, transfection or transduction can involve any method known to a skilled person for introducing nucleic acid molecules into host cells (e.g., mammalian cells, such as HEK293). For example, transfection can include vector-based transfection, such as the use of one or more vectors (e.g., plasmids). Transfection methods can also include, but are not limited to, viral-based transfection, electroporation, lipofection (e.g., with cationic lipids and/or liposomes), calcium phosphate precipitation, nanoparticle-based transfection, transfection based on cationic polymers (e.g., DEAE-dextran or polyethylenimine), or a combination of any of the foregoing methods. A number of transfection techniques are generally known in the art (See, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a payload as described herein, into suitable host cells.

In some embodiments, host cells are transfected with a transfection reagent. In some embodiments, a transfection reagent comprises a polymer (e.g., polyethylenimine (PEI)), calcium phosphate, a lipid capable of traversing a cell membrane (e.g., a liposome or a micelle), or a nanoparticle. In some embodiments, host cells are transfected with PEI. In some embodiments, host cells are transfected with a weight (wt.) ratio of DNA to transfection reagent (e.g., PEI) of about 1:1 to about 1:2, about 1:1 to about 1:5, or about 1:1 to about 1:10, e.g., about 1:0.05, about 1:1, about 1:1.25, about 1:1.5, about 1:2, about 1:2.5, about 1:3, about 1:3.5, about 1:4, about 1:4.5, about 1:5, about 1:6, about 1:7, about 1:8, about 1:9, or about 1:10. In some embodiments, a wt. ratio of DNA to transfection reagent is dependent on cell culture density (e.g., of adherent or suspension host cells).

In some embodiments, transfection complexes are formed between a transfection reagent (e.g., PEI) and one, two, or three of: (i) a first vector encoding at least one Adenovirus 2 (Ad2) helper polypeptides; (ii) a second vector encoding at least one Rep polypeptide and/or at least one Cap polypeptide; and (iii) a third vector encoding at least one payload. In some embodiments, transfection complexes are formed between a transfection reagent and one or two of: (i) a first vector encoding at least one Ad2 helper function polypeptides and at least one Rep polypeptide, and (ii) a second vector encoding at least one Cap polypeptide and at least one payload. In some embodiments, transfection complexes are formed for less than 10 minutes+/−15%, e.g., for 9 minutes+/−15%, 8 minutes+/−15%, 7 minutes+/−15%, 6 minutes+/−15%, 5 minutes+/−15%, 4 minutes+/−15%, 3 minutes+/−15%, 2 minutes+/−15%, 1 minute+/−15%, or less.

In some embodiments, a vector mass ratio of: (i) a first vector encoding at least one Ad2 helper polypeptide to (ii) a second vector encoding at least one Rep polypeptide and/or at least one Cap polypeptide to (iii) a third vector encoding at least one payload is used in the methods, e.g., for transfection. In some embodiments, a vector mass ratio of: (i) a first vector encoding at least one Ad2 helper polypeptide to (ii) a second vector encoding at least one Rep polypeptide and/or at least one Cap polypeptide to (iii) a third vector encoding at least one payload is about 1:1:1. In some embodiments, a vector mass ratio of: (i) a first vector encoding at least one Ad2 helper polypeptide to (ii) a second vector encoding at least one Rep polypeptide and/or at least one Cap polypeptide to (iii) a third vector encoding at least one payload is not about 1:1:1. In some embodiments, a vector mass ratio of (i) a first vector encoding at least one Ad2 helper polypeptide to (ii) a second vector encoding at least one Rep polypeptide and/or at least one Cap polypeptide to (iii) a third vector encoding at least one payload is about 1:0.5:1, about 1:1:1, about 2:1:1, about 3:1:1, about 4:1:1, about 5:1:1, about 6:1:1, about 7:1:1, about 8:1:1, about 9:1:1, about 10:1:1, about 1:10:5, about 2:0.5:1, about 10:0.5:1, about 5:0.5:1, about 1:5:0.5, about 20:10:1, about 1:2:1, about 1:3:1, about 1:4:1, about 1:5:1, about 1:6:1, about 1:7:1, about 1:8:1, about 1:9:1, about 1:10:1, about 1:1:10, about 1:1:9, about 1:1:8, about 1:1:7, about 1:1:6, about 1:1:5, about 1:1:4, about 1:1:3, about 1:1:2, or about 1:0.5:5.

In some embodiments, a vector molar ratio of (i) a first vector encoding at least one Ad2 helper polypeptide to (ii) a second vector encoding at least one Rep polypeptide and/or at least one Cap polypeptide to (iii) a third vector encoding at least one payload is: about 10.8 to about 8.2 to about 1, or about 10.5-11 to about 8-8.4 to about 1. In some embodiments, a vector molar ratio of (i) a first vector encoding at least one Ad2 helper polypeptide to (ii) a second vector encoding at least one Rep polypeptide and/or at least one Cap polypeptide to (iii) a third vector encoding at least one payload is: about 2 to about 1 to about 0.5.

Culturing

The present disclosure, among other things, provides methods for culturing of a host cell with at least one vector described herein for production of rAAV particles. A wide variety of growth media (e.g., mammalian growth media) may be used in accordance with the present invention. In certain embodiments, cells may be grown in one of a variety of chemically defined media, wherein the components of the media are both known and controlled. In certain embodiments, cells may be grown in a complex medium, in which not all components of the medium are known and/or controlled.

A culture of host cells can be prepared in any medium suitable for a particular cell type being cultured. In some embodiments, a host cell medium comprises, e.g., inorganic salts, carbohydrates (e.g., sugars, such as glucose, galactose, maltose, or fructose), amino acids, vitamins (e.g., B group vitamins (e.g., B12), vitamin A, vitamin E, riboflavin, thiamine, or biotin), fatty acids (e.g., cholesterol or steroids), proteins (e.g., albumin, transferrin, fibronectin, or fetuin), serum (e.g., albumins, growth factors, or growth inhibitors, such as, fetal bovine serum, newborn calf serum, or horse serum), trace elements (e.g., zinc, copper, selenium, or tricarboxylic acid intermediates), hydrolysates (e.g., derived from plant or animal sources), or combinations thereof.

Commercially available media can be used for culturing host cells described herein. Exemplary media can include, but is not limited to, Dulbecco's Modified Eagle's Medium ([DMEM], Sigma), FreeStyle™ F17 Expression Medium (ThermoFisher), DMEM/F12 medium (Invitrogen), CD OptiCHO™ medium (Invitrogen), CD EfficientFeed™ media. (Invitrogen), Cell Boost (HyClone™) media (GE Life Sciences), BalanCD™ CHO Feed (Irvine Scientific), BD Recharge™ (Becton Dickinson), Cellvento Feed™ (EMD Millipore), Ex-cell CHOZN Feed™ (Sigma-Aldrich), CHO Feed Bioreactor Supplement (Sigma-Aldrich), SheffCHO™ (Kerry), Zap-CHO™ (Invitria), ActiCHO™ (PAA/GE Healthcare), Minimal Essential Medium (Sigma), or RPMI-1640 (Sigma). Media can be supplemented as necessary with hormones and/or other growth factors (e.g., insulin, transferrin, or epidermal growth factor), salts (e.g., sodium chloride, calcium, magnesium, or phosphate), buffers (e.g., HEPES), nucleosides (e.g., adenosine or thymidine), antibiotics (e.g., kanamycin, puromycin, neomycin, hygromycin, blasticidin, gentamycin, Gr18, or zeocin), trace elements, lipids (e.g., linoleic or other fatty acids), or glucose or an equivalent energy source. In some embodiments, the media for culturing host cells comprises glutamine or a glutamine dipeptide. In some embodiments, the media for culturing host cells comprises a surfactant. In some embodiments, the nutrient media is serum-free media, a protein-free media, or a chemically defined media. Any other necessary supplements can also be included at appropriate concentrations that would be known to those skilled in the art.

After culturing of host cells as described herein, a plurality of rAAV particles are recovered. In some embodiments, rAAV particles are recovered by lysing host cells and recovering rAAV particles from lysate, e.g., after centrifugation. In some embodiments, rAAV particles are recovered from culture supernatant. In some embodiments, a lysis solution for host cells comprises chemical reagents, e.g., detergents (e.g., sodium dodecyl sulfate (SDS), ethyl trimethyl ammonium bromide, Triton X-100, bile salts, such as cholate, or zwitterionic detergents, such as CHAPS). In some embodiments, a lysis solution for host cells comprises a salt (e.g., NaCl) and a high pH (e.g., a pH of greater than about 7). In some embodiments, rAAV particles are purified using purification methods, such as chromatography (e.g., affinity chromatography or ion-exchange chromatography (e.g., cation exchange chromatography)) or filtration (e.g., UF/DF filtration)).

In some embodiments, a plurality of rAAV particles are produced in a large-scale preparation. In some embodiments, a large-scale preparation of host cells (e.g., suspension host cells) is at least 10 liters+/−15% of culture media, e.g., between 50 liters+/−15% to 1000 liters+/−15% of culture media or between 50 liters+/−15% to 2000 liters+/−15% of culture media, e.g., at least 20 liters+/−15%, 30 liters+/−15%, 40 liters+/−15%, 50 liters+/−15%, 55 liters+/−15%, 60 liters+/−15%, 65 liters+/−15%, 70 liters+/−15%, 75 liters+/−15%, 80 liters+/−15%, 85 liters+/−15%, 90 liters+/−15%, 95 liters+/−15%, 100 liters+/−15%, 200 liters+/−15%, 300 liters+/−15%, 400 liters+/−15%, 500 liters+/−15%, 600 liters+/−15%, 700 liters+/−15%, 800 liters+/−15%, 900 liters+/−15%, 1,000 liters+/−15%, 1,250 liters+/−15%, 1,500 liters+/−15%, 1,750 liters+/−15%, 2,000 liters+/−15%, or more of culture media.

In some embodiments, a large-scale preparation of host cells (e.g., adherent host cells) is at least 5 m²+/−15% of culture media, e.g., between 5 m²+/−15% to 500 m²+/−15% of culture media, e.g., at least 5 m²+/−15%, 10 m²+/−15%, 15 m²+/−15%, 20 m²+/−15%, 25 m²+/−15%, 20 m²+/−15%, 35 m²+/−15%, 40 m²+/−15%, 45 m²+/−15%, 50 m²+/−15%, 55 m²+/−15%, 60 m²+/−15%, 65 m²+/−15%, 75 m²+/−15%, 80 m²+/−15%, 85 m²+/−15%, 90 m²+/−15%, 95 m²+/−15%, 100 m²+/−15%, 150 m²+/−15%, 175 m²+/−15%, 200 m²+/−15%, 225 m²+/−15%, 250 m²+/−15%, 275 m²+/−15%, 300 m²+/−15%, 325 m²+/−15%, 330 m²+/−15%, 340 m²+/−15%, 350 m²+/−15%, 375 m²+/−15%, 400 m²+/−15%, 425 m²+/−15%, 450 m²+/−15%, 475 m²+/−15%, 500 m²+/−15%, 600 m²+/−15%, 700 m²+/−15%, 800 m²+/−15%, 900 m²+/−15%, 1000 m²+/−15%, 1100 m²+/−15%, 1200 m²+/−15%, 1300 m²+/−15%, 1400 m²+/−15%, 1500 m²+/−15%, 1600 m²+/−15%, 1700 m²+/−15%, 1800 m²+/−15%, 1900 m²+/−15%, 2000 m²+/−15%, 2500 m²+/−15%, 3000 m²+/−15%, 3500 m²+/−15%, 4000 m²+/−15%, 4500 m²+/−15%, 5000 m²+/−15%, 5500 m²+/−15%, 6000 m²+/−15%, 6500 m²+/−15%, 7000 m²+/−15%, 7500 m²+/−15%, 8000 m²+/−15%, 8500 m²+/−15%, 9000 m²+/−15%, 9500 m²+/−15%, 10,000 m²+/−15%, 11,000 m²+/−15%, 12,000 m²+/−15%, 13,000 m²+/−15%, 14,000 m²+/−15%, 15,000 m²+/−15%, or more of culture media.

A host cell can be cultured in a cell culture vessel or a bioreactor. Cell culture vessels include a cell culture dish, plate or flask. In some embodiments, a cell culture vessel is suitable for/used for culturing adherent cells. In other embodiments, a cell culture vessel is suitable for/used for culturing suspension cells. Exemplary cell culture vessels include 35 mm, 60 mm, 100 mm, or 150 mm dishes, multi-well plates (e.g., 6-well, 12-well, 24-well, 48-well, or 96 well plates), or flasks (e.g., T-flasks, e.g., T-25, T-75, or T-160 flasks), or shaker flasks.

In some embodiments, a host cell is cultured in a bioreactor. In some embodiments, a bioreactor is suitable for/used for culturing adherent cells. In some embodiments, host cells are cultured in one or more HYPERStack®—36 culture vessels. In some embodiments, host cells are cultured in one or more CellSTACK® culture vessels. In other embodiments, a bioreactor is suitable for/used for culturing suspension cells. A bioreactor can be, e.g., a continuous flow batch bioreactor, a perfusion bioreactor, a batch process bioreactor, or a fed batch bioreactor. An exemplary bioreactor is a fixed bed bioreactor, e.g., an iCELLis bioreactor (used for culturing adherent cells). A bioreactor can be maintained under conditions sufficient to produce rAAV particles. Culture conditions can be modulated to optimize yield, purity, or structure of rAAV particles.

In some embodiments, a bioreactor comprises a plurality of host cells, e.g., at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ host cells. In some embodiments, a bioreactor comprises between 1×10⁷ to 1×10¹⁴ host cells; between 1×10⁷ to 0.5×10¹⁴ host cells; between 1×10⁷ to 1×10¹³ host cells; between 1×10⁷ to 0.5×10¹³ host cells; between 1×10⁷ to 1×10¹² host cells; between 1×10⁷ to 0.5×10¹² host cells; between 1×10⁷ to 1×10¹¹ host cells; between 1×10⁷ to 0.5×10¹¹ host cells; between 1×10⁷ to 1×10¹⁰ host cells; between 1×10⁷ to 0.5×10¹⁰ host cells; between 1×10⁷ to 1×10⁹ host cells; between 1×10⁷ to 0.5×10⁹ host cells; between 1×10⁷ to 1×10⁸ host cells; between 1×10⁷ to 0.5×10⁸ host cells; between 0.5×10⁸ to 1×10¹⁴ host cells; between 1×10⁸ to 1×10¹⁴ host cells; between 0.5×10⁹ to 1×10¹⁴ host cells; between 1×10⁹ to 1×10¹⁴ host cells; between 0.5×10¹⁰ to 1×10¹⁴ host cells; between 1×10¹⁰ to 1×10¹⁴ host cells; between 0.5×10¹¹ to 1×10¹⁴ host cells; between 1×10¹¹ to 1×10¹⁴ host cells; between 0.5×10¹² to 1×10¹⁴ host cells; between 1×10¹² to 1×10¹⁴ host cells; between 0.5×10¹³ to 1×10¹⁴ host cells; between 1×10¹³ to 1×10¹⁴ host cells; or between 0.5×10¹³ to 1×10¹⁴ host cells.

In some embodiments, a bioreactor comprises at least 10 liters of culture media, e.g., between 50 liters to 1000 liters of culture media, e.g., at least 20 liters, 30 liters, 40 liters, 50 liters, 55 liters, 60 liters, 65 liters, 70 liters, 75 liters, 80 liters, 85 liters, 90 liters, 95 liters, 100 liters, 200 liters, 300 liters, 400 liters, 500 liters, 600 liters, 700 liters, 800 liters, 900 liters, or 1,000 liters of culture media. In some embodiments, a bioreactor comprises at least 5 m² of culture media, e.g., between 5 m² to 500 m² of culture media, e.g., at least 5 m², 10 m², 15 m², 20 m², 25 m², 20 m², 35 m², 40 m², 45 m², 50 m², 55 m², 60 m², 65 m², 75 m², 80 m², 85 m², 90 m², 95 m², 100 m², 150 m², 175 m², 200 m², 225 m², 250 m², 275 m², 300 m², 325 m², 330 m², 340 m², 350 m², 375 m², 400 m², 425 m², 450 m², 475 m², or 500 m² of culture media.

In an embodiment, a bioreactor is maintained under conditions that promote growth of a host cell, e.g., at a temperature (e.g., 37° C.) and gas concentration (e.g., 5%-10% CO₂) that is permissive for growth of the host cell. For example, a bioreactor can perform one or more of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), cleaning, and/or sterilization. Exemplary bioreactor units, may contain multiple reactors within a unit, e.g., a unit can comprise 1, 2, 3, 4, 5, 10, 15, 20, 25, 30, 35, 40, 45, 50, 60, 70, 80, 90, or 100, or more bioreactors. Any suitable bioreactor diameter and/or shape can be used. In some embodiments, suitable reactors can be round, e.g., cylindrical. In some embodiments, suitable reactors can be square, e.g., rectangular.

rAAV Particle Production

The present disclosure, among other things, rAAV particles produced using methods described herein. Generally, rAAV particles produced using methods described herein may be of any AAV serotype. AAV serotypes generally have different tropisms to infect different tissues. In some embodiments, an AAV serotype is selected based on a tropism.

In some embodiments, an AAV particle may comprise or be based on a serotype selected from any of the following serotypes, and variants thereof, including, but not limited to: AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb. 1, AAV29.5/bb.2, AAV2G9, AAV-2-pre-miRNA-101, AAV3, AAV3.1/hu.6, AAV3.1/hu.9, AAV3-1 1/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAV A3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu. 1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu68, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC 8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63 AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8.

In certain embodiments, an AAV particle comprises an AAV2 serotype or a variant thereof. In certain embodiments, an AAV particle comprises an AAV5 serotype or a variant thereof. In certain embodiments, an AAV particle comprises an AAV8 serotype or a variant thereof. In certain embodiments, an AAV particle comprises an AAV9 serotype or a variant thereof. In certain embodiments, an AAV particle comprises AAVhu68 or a variant thereof. In certain embodiments, an AAV particle comprises AAVrh10 or a variant thereof.

In some embodiment, a plurality of rAAV particles are produced with methods described herein at a higher titer, e.g., such there is improved rAAV particle production. In some embodiments, the improved production comprises a higher yield of the plurality of rAAV particles relative to a plurality of rAAV particles produced with a helper vector comprising a nucleic acid sequence of an antibiotic resistance gene other than KanR (e.g., an Ampicillin resistance gene). In some embodiments, a high titer is relative to AAV particles produced from a reference helper vector (e.g., an Ad5 vector, e.g., an Ad5 vector described herein), e.g., under otherwise identical conditions.

In some embodiments, a high titer of rAAV particles is greater than 2.6×10¹⁰ vg/cm²+/−15%, e.g., when cultured in a flask and/or in an adherent culture. In some embodiments, a high titer is greater than about 2.6×10¹⁰ viral genomes (vg)/cm², e.g., greater than about 2.7×10¹⁰ vg/cm², about 2.8×10¹⁰ vg/cm², about 2.9×10¹⁰ vg/cm² about 3.0×10¹⁰ vg/cm², or greater, e.g., when cultured in a flask (e.g., a flask described herein, e.g., a t-flask) and/or in an adherent culture. In some embodiments, a high titer is greater than 7.0×10⁹ vg/mL+/−15%, e.g., when cultured in suspension. In some embodiments, a high titer is greater than about 7.0×10⁹ vg/mL, e.g., greater than about 7.5×10⁹ vg/mL, 8.0×10⁹ vg/mL, 8.5×10⁹ vg/mL, 9.0×10⁹ vg/mL, 1.0×10¹⁰ vg/mL, 1.5×10¹⁰ vg/mL, 2.0×10¹⁰ vg/mL, 2.5×10¹⁰ vg/mL, 3.0×10¹⁰ vg/mL, 3.5×10¹⁰ vg/mL, 4.0×10¹⁰ vg/mL, 4.5×10¹⁰ vg/mL, 5.0×10¹⁰ vg/mL, 5.5×10¹⁰ vg/mL, 6.0×10¹⁰ vg/mL, 6.5×10¹⁰ vg/mL, 7.0×10¹⁰ vg/mL, 7.5×10¹⁰ vg/mL, 8.0×10¹⁰ vg/mL, 8.5×10¹⁰ vg/mL, 9.0×10¹⁰ vg/mL, 9.5×10¹⁰ vg/mL, 1.0×10¹¹ vg/mL, 1.5×10¹¹ vg/mL, 2.0×10¹¹ vg/mL, or higher, e.g., when cultured in suspension.

In some embodiments, a high titer of rAAV particles is at least about 7.0×10⁹ vg/cm² (e.g., at least about 7.0×10⁹, about 7.5×10⁹, about 8.0×10⁹, about 8.5×10⁹, about 9.0×10⁹, about 9.5×10⁹, about 1.0×10¹⁰, about 1.5×10¹⁰, about 2.0×10¹⁰, about 2.5×10¹⁰, about 3.0×10¹⁰, about 3.5×10¹⁰, about 4.0×10¹⁰ vg/cm², or higher), e.g., when cultured in an adherent culture. In some embodiments, a high titer of rAAV particles is at least about 2.5×10¹⁰ vg/cm², about 2.75×10¹⁰ vg/cm², about 3.0×10¹⁰ vg/cm², about 3.5×10¹⁰ vg/cm², about 4.0×10¹⁰ vg/cm² vg/cm², or higher, e.g., when cultured in a flask. In some embodiments, a high titer of rAAV particles is at least about 7.0×10⁹ vg/cm², about 7.5×10⁹ vg/cm², about 8.0×10⁹ vg/cm², about 8.5×10⁹ vg/cm², about 9.0×10⁹ vg/cm², about 9.5×10⁹ vg/cm², about 1.0×10¹⁰ vg/cm², about 1.5×10¹⁰ vg/cm², or higher, e.g., when cultured in a fixed bed bioreactor.

In some embodiments, a high titer of rAAV particles is greater than 5.0×10¹³ vg/m²+/−15%, e.g., greater than 6.0×10¹³ vg/m²+/−15, 7.0×10¹³ vg/m²+/−15, 8.0×10¹³ vg/m²+/−15, 9.0×10¹³ vg/m²+/−15, 1.0×10¹⁴ vg/m²+/−15, 2.0×10¹⁴ vg/m²+/−15, 3.0×10¹⁴ vg/m²+/−15, 4.0×10¹⁴ vg/m²+/−15, 5.0×10¹⁴ vg/m²+/−15, 6.0×10¹⁴ vg/m²+/−15, 7.0×10¹⁴ vg/m²+/−15, 8.0×10¹⁴ vg/m²+/−15, 9.0×10¹⁴ vg/m²+/−15, or more.

In some embodiments, a plurality of rAAV particles described herein is harvested after at least 3 days of culturing. In some embodiments, a plurality of rAAV particles described herein is harvested after at least about 3 days to about 10 days of culturing, e.g., about 3 days to about 7 days, about 3 days to about 5 days, about 4 days to about 9 days, about 4 days to about 8 days, or about 4 days to about 6 days of culturing, e.g., after at least about 3 days, about 4 days, about 5 days, about 6 days, about 7 days, about 8 days, about 9 days, about 10 days, or longer after culturing. In some embodiments, a plurality of rAAV particles produced with methods described herein is substantially free of one or both of a helper adenovirus or a herpes virus. In some embodiments, a plurality of rAAV particles is substantially free of one or both of a helper adenovirus or a herpes virus, e.g., a purity of at least about 90%, about 91%, about 92%, about 93%, about 94%, about 95%, about 96%, about 97%, about 98%, about 99%, or more free of one or both of a helper adenovirus or a herpes virus. In some embodiments, a plurality of rAAV particles has a reduced level of adenoviral impurities, e.g., a purity of at least about 50%, about 60%, about 70%, about 80%, about 85%, about 90%, about 91%, about 92%, about 93%, about 94%, about 95%, about 96%, about 97%, about 98%, about 99%, or more free of adenoviral impurities, e.g., relative to rAAV particles produced with a reference helper vector (e.g., an Ad5 vector, an Ad5 vector described herein). In some embodiments, a plurality of rAAV particles comprises less than or about 50% adenoviral impurities, e.g., less than or about 45%, 40%, 35%, 30%, 25%, 20%, 15%, 10%, 5%, 4%, 3%, 2%, 1%, or less adenoviral impurities, e.g., relative to rAAV particles produced with a reference helper vector (e.g., an Ad5 vector, e.g., an Ad5 vector described herein).

In some embodiments, a plurality of rAAV particles produced with methods described herein comprises increased expression of at least one payload, e.g., relative to rAAV particles produced with a reference helper vector (e.g., an Ad5 vector, e.g., an Ad5 vector described herein). In some embodiments, a plurality of rAAV expresses or comprises at least one payload at a level of about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, or more greater than rAAV particles produced with a reference helper vector (e.g., an Ad5 vector, e.g., an Ad5 vector described herein)).

In some embodiments, a plurality of rAAV particles produced with methods described herein has improved infectivity of a cell or a tissue, e.g., relative to rAAV particles produced with a reference helper vector (e.g., an Ad5 vector, e.g., an Ad5 vector described herein)). In some embodiments, a cell or a tissue infected with a plurality of rAAV particles comprises an increased amount or expression of at least one payload, e.g., an increase of at least about 1%, about 2%, about 3%, about 4%, about 5%, about 10%, about 15%, about 20%, about 25%, about 30%, about 40%, about 45%, about 50%, about 60%, about 70%, about 80%, about 85%, about 90%, about 91%, about 92%, about 93%, about 94%, about 95%, about 96%, about 97%, about 98%, about 99%, or more, e.g., relative to rAAV particles produced with a reference helper vector (e.g., an Ad5 vector, e.g., an Ad5 vector described herein)).

In some embodiments, a plurality of rAAV particles produced with methods described herein has improved transduction (e.g., improved transduction efficiency), e.g., relative to rAAV particles produced with a reference helper vector (e.g., an Ad5 vector)). In some embodiments, transduction efficiency is increased by at least about 1%, about 2%, about 3%, about 4%, about 5%, about 10%, about 15%, about 20%, about 25%, about 30%, about 40%, about 45%, about 50%, about 60%, about 70%, about 80%, about 85%, about 90%, about 91%, about 92%, about 93%, about 94%, about 95%, about 96%, about 97%, about 98%, about 99%, or more, e.g., relative to rAAV particles produced with a reference helper vector (e.g., an Ad5 vector, e.g., an Ad5 vector described herein)).

The foregoing methods for producing recombinant vectors are not meant to be limiting, and other suitable methods will be apparent to the skilled artisan.

Compositions

The present disclosure, among other things, provides a composition comprising a plurality of rAAV particles formed by methods described herein. In some embodiments, a composition comprises a pharmaceutical composition comprising at least one pharmaceutically acceptable component (e.g., a pharmaceutically acceptable carrier, diluent, or excipient). Such pharmaceutical compositions are useful for, among other things, administration to a subject in vivo or ex vivo.

In some embodiments, pharmaceutical compositions also contain a pharmaceutically acceptable carrier, excipient, or diluent. Such excipients include any pharmaceutical agent, e.g., a pharmaceutical agent that does not itself induce an immune response harmful to the individual receiving the composition, and which may be administered without undue toxicity. Pharmaceutically acceptable excipients include, but are not limited to, liquids, such as water, saline, glycerol, sugars, and ethanol. Pharmaceutically acceptable salts can also be included therein, for example, mineral acid salts, such as hydrochlorides, hydrobromides, phosphates, or sulfates; and the salts of organic acids, such as acetates, propionates, malonates, or benzoates. Additionally, auxiliary substances, such as wetting or emulsifying agents or pH buffering substances, may be present in such vehicles.

Pharmaceutical compositions may be provided as a salt and can be formed with many acids, including but not limited to, hydrochloric, sulfuric, acetic, lactic, tartaric, malic, or succinic. Salts tend to be more soluble in aqueous or other protonic solvents than corresponding free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, and isotonic and absorption promoting or delaying agents, compatible with pharmaceutical administration or in vivo contact or delivery. Aqueous and non-aqueous solvents, solutions, and suspensions may include suspending agents and thickening agents. Such pharmaceutically acceptable carriers include tablets (coated or uncoated), capsules (hard or soft), microbeads, powder, granules, and crystals. Supplementary active compounds (e.g., preservatives, antibacterial, antiviral, and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable, or synthetic oils. Aqueous injection suspensions may contain substances that increase the viscosity of a suspension, such as sodium carboxymethyl cellulose, sorbitol, or dextran. Additionally, suspensions may be prepared as appropriate oil injection suspensions. Suitable lipophilic solvents or vehicles include fatty oils, such as sesame oil, or synthetic fatty acid esters, such as ethyl oleate or triglycerides, or liposomes. Optionally, the suspension may also contain suitable stabilizers or agents that increase solubility to allow for preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, Pa. Lippincott Williams & Wilkins, 2005).

Administration

The present disclosure, among other things, provides methods of administering a composition (e.g., a pharmaceutical composition) comprising a plurality of rAAV particles formed by methods described herein. Compositions (e.g., pharmaceutical compositions) comprising rAAVs produced with the methods described herein can be used to treat any disease or disorder, e.g., subjects suffering from or susceptible to a disease or disorder described herein. The route and/or mode of administration can vary depending upon the desired results. One with skill in the art (e.g., a physician), is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. Mode of administration is left to discretion of a practitioner.

For example, a composition may be administered by retinal, subretinal, intravitreal, or suprachoroidal injection or infusion. Additional exemplary routes of administration may include, but are not limited to, bronchial (e.g., bronchial instillation), buccal, enteral, interdermal, intra-arterial, intracisterna magna (ICM), intradermal, intragastric, intramedullary, intramuscular, intranasal, intra-parenchymal (e.g., intra-thalamic), intraperitoneal, intrathecal, intravenous, intraventricular, mucosal, nasal, oral, rectal, intraspinal, spinal sub-pial, subcutaneous, sublingual, topical, tracheal (e.g., intratracheal instillation), transdermal, vaginal, and vitreal administration.

Methods and uses disclosed herein include delivery and administration systemically, regionally or locally, or by any route, for example, by injection or infusion. A composition (e.g., pharmaceutical composition) comprising a plurality of rAAV particles formed by methods described herein may be administered by injection or infusion by any route. For example, a composition may be administered by retinal, subretinal, intravitreal, suprachoroidal, intraspinal, intracisterna magna, or intrathecal injection or infusion.

Delivery of a pharmaceutical composition in vivo may generally be accomplished via injection using a conventional syringe, although other delivery methods such as convection-enhanced delivery can also be used. For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in treatment of patients with certain diseases or disorders may determine the optimal route for administration of vectors described herein.

The disclosure provides methods for introducing a composition (e.g., a pharmaceutical composition) comprising rAAV particles described herein into a cell, a tissue, or an animal. In some embodiments, such methods comprise contacting a cell, a tissue, or an animal with a composition comprising rAAV particles described herein, such that at least one payload is expressed or present in the cell, tissue, or animal.

The disclosure also provides methods for administering a composition (e.g., a pharmaceutical composition) comprising rAAV particles described herein to a subject. In some embodiments, such methods include administering to a subject (e.g., a mammal), a composition comprising rAAV particles described herein, such that at least one payload is expressed or present in the subject (e.g., in a cell or tissue of a subject). In some embodiments, a method includes providing cells of a subject (e.g., a mammal) with a composition (e.g., a pharmaceutical composition) comprising rAAV particles described herein, such that at least one payload is expressed or present in the subject.

A composition (e.g., a pharmaceutical composition) comprising rAAV particles described herein can be administered in a sufficient or effective amount to a subject in need thereof. Doses can vary and depend upon a type, onset, progression, severity, frequency, duration, or probability of disease to which treatment is directed, the clinical endpoint desired, previous or simultaneous treatments, the general health, age, gender, race or immunological competency of the subject, and other factors that will be appreciated by a skilled artisan. Dose amount, number, frequency, or duration may be proportionally increased or reduced, as indicated by any adverse side effects, complications, or other risk factors of treatment and status of the subject. A skilled artisan will appreciate the factors that may influence the dosage and timing required to provide an amount sufficient for providing a therapeutic or prophylactic benefit.

A dose to achieve a therapeutic effect will vary based on several factors including, but not limited to: route of administration, level of payload or payload expression required to achieve a therapeutic effect, specific disease treated, any host immune response, and stability of payload or payload expression. One skilled in the art can determine a dose range to treat a patient having a particular disease or disorder based on the aforementioned factors, as well as other factors.

An effective amount or a sufficient amount can (but need not) be provided in a single administration, may require multiple administrations, and, can (but need not) be, administered alone or in combination with another composition. For example, an amount may be proportionally increased as indicated by need of a subject, type, status, and severity of disease treated or side effects (if any) of treatment. Amounts considered effective also include amounts that result in a reduction of use of another treatment, therapeutic regimen, or protocol.

Accordingly, pharmaceutical compositions include compositions comprising rAAV particles in an effective amount to achieve an intended therapeutic purpose. Determining a therapeutically effective dose is well within the capability of a skilled medical practitioner using techniques and guidance provided herein. Therapeutic doses can depend on, among other factors, age and general condition of a subject, severity of a disease or disorder, and payload amount or expression in a subject. Thus, a therapeutically effective amount in humans will fall in a relatively broad range that may be determined by a medical practitioner based on response of an individual patient to rAAV-based treatment. Pharmaceutical compositions may be delivered to a subject so as to allow production of a payload described herein in vivo by gene- and or cell-based therapies or by ex-vivo modification of a patient's or donor's cells.

In some embodiments, a composition (e.g., a pharmaceutical composition) comprising rAAV particles described herein may be administered to a subject once daily, weekly, every 2, 3, or 4 weeks, or even at longer intervals. In some embodiments, a composition (e.g., a pharmaceutical composition) comprising rAAV particles described herein may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments, a composition (e.g., a pharmaceutical composition) comprising rAAV particles described herein may be administered (i) one or more times during an initial time period of up to 2, 4, or 6 weeks or less; followed by (ii) a period of no administration of, e.g., 1, 2, 3, 4, 5, 6, 7, 8, 9, or 10 years. In some embodiments, a subject is monitored before and/or following treatment with a composition (e.g., a pharmaceutical composition) comprising rAAV particles described herein.

Uses

The present disclosure, among other things, provides methods of delivering a gene therapy as described herein to a cell or tissue. The present disclosure also provides methods of treating a subject with a composition (e.g., a pharmaceutical composition) comprising a plurality of rAAV particles as described herein.

In some embodiments, methods and kits of the present invention may be used for the evaluation and/or monitoring of gene therapy. In some embodiments, gene therapy comprises administration of a composition (e.g., a pharmaceutical composition) comprising a plurality of rAAV particles described herein. In some embodiments, samples for evaluating and/or monitoring gene therapy may be obtained prior to the initiation of gene therapy. In some embodiments, samples are obtained after a first gene therapy treatment or dose. In some embodiments, samples are obtained after the conclusion of gene therapy. In some embodiments, samples are obtained at specific time points, intervals, or any other metric of time before, during, or after gene therapy is performed.

In some embodiments, a composition (e.g., a pharmaceutical composition) comprising a plurality of rAAV particles as described herein is administered to a subject suffering from or at risk of a disease, disorder, or condition. In some embodiments, a composition (e.g., a pharmaceutical composition) comprising a plurality of rAAV particles as described herein is administered in combination with one or more additional therapeutics agents to a subject. In some embodiments, a composition (e.g., a pharmaceutical composition) comprising a plurality of rAAV particles as described herein is contacted with an organ, tissue, or cells ex vivo. The organ, tissue, or cells can be introduced into a subject and can be protected from damage that would otherwise be caused by the recipient's immune system.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following example. An example is provided for illustrative purposes only. It is not to be construed as limiting the scope or content of the disclosure in any way.

### EXAMPLE

The following example is put forth so as to provide those of ordinary skill in the art with a complete disclosure and description of how to make and use the present invention, and is not intended to limit the scope of what the inventors regard as their invention nor are they intended to represent that the experiments below are all or the only experiments performed.

### Example 1: Production of rAAV Using Ad2 Helper Plasmid

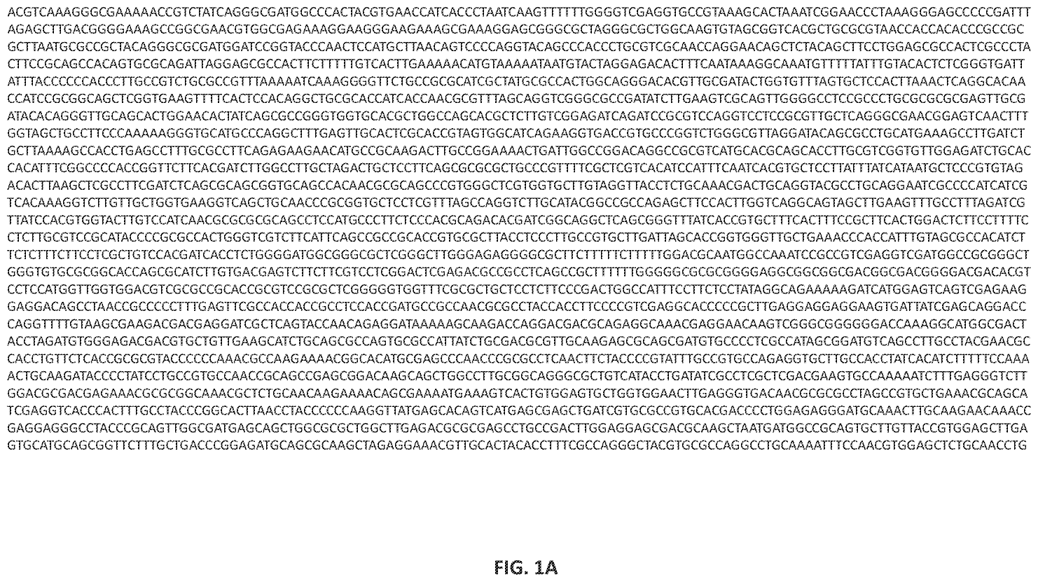

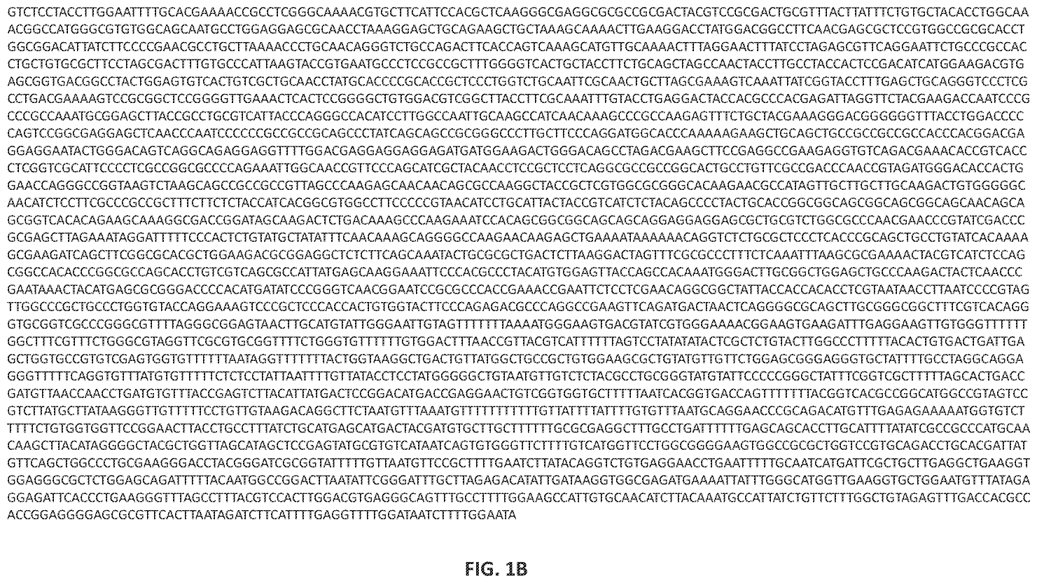

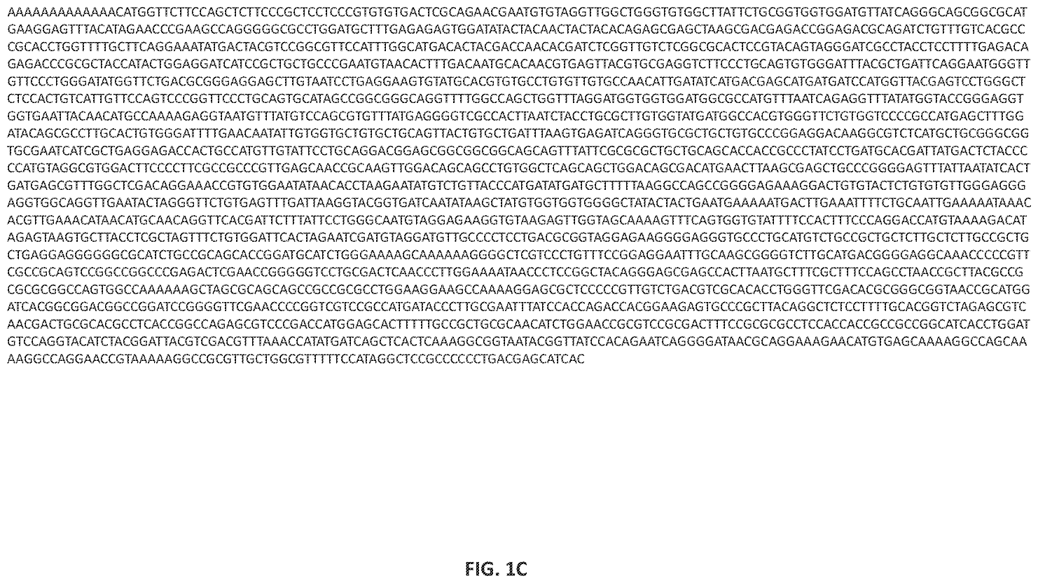

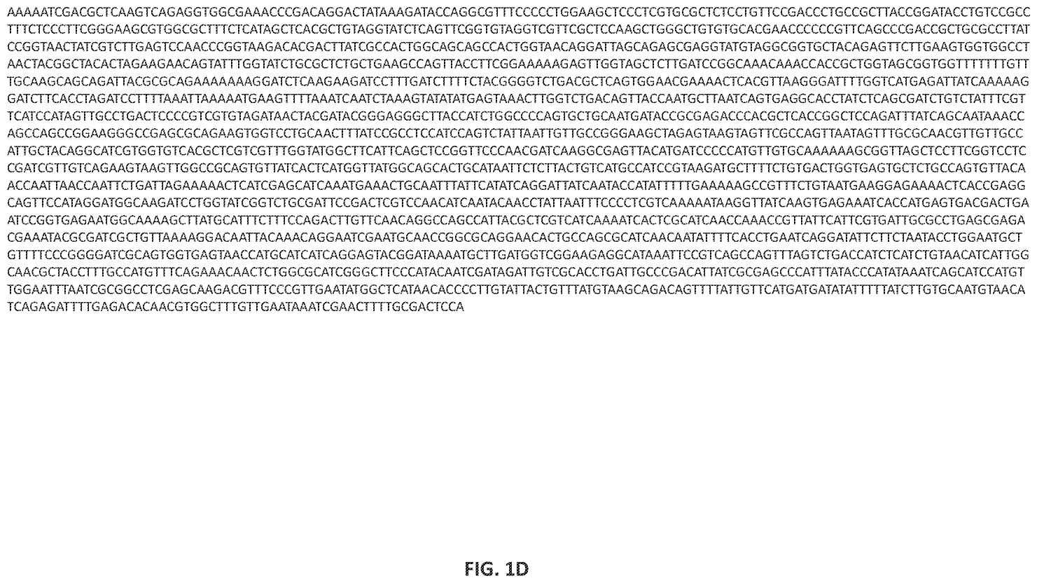

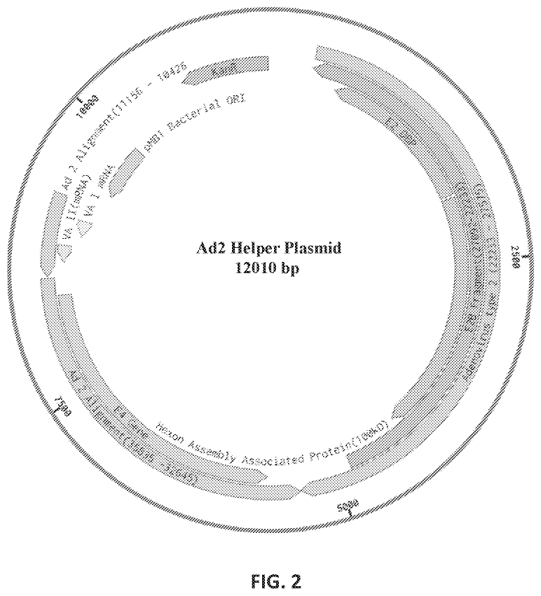

An Adenovirus 2 (Ad2) helper plasmid was generated that provides adenoviral helper genes necessary to generate functional AAV particles (E2A DBP, E4, and VA RNA) when transfected into an E1 complementing cell line, such as HEK293. A plasmid encoding Ad2 helper polypeptides (SEQ ID NO: 1; hereinafter “A2 helper plasmid”) was derived from the Adenovirus 2 genome (Genbank Accession No. J01917.1) (FIGS. 1A-1D). The nucleic acid sequence of SEQ ID NO: 1 is 12,010 bp in length. VA RNA, E4, and E2A Helper polypeptides are oriented from 5′ to 3′ in direction on the Ad2 helper plasmid (FIG. 2).

The Ad2 helper plasmid was demonstrated to have improved productivity in terms of AAV production in host cells in: (i) an adherent transient transfection process in both t-flask and an iCELLis® Nano Bioreactor compared to two Ad5 helper plasmids (pAd5-1 and pAd5-2, which are each derived from the Adenovirus 5 genome (Genbank Accession No. AY601635)), and (ii) in a suspension transient transfection process in shaker flasks compared to an Ad5 helper plasmid (pAd5-1). The Ad2 helper plasmid was evaluated using transient transfection with adherent HEK293 host cells and suspension HEK293 cells. Adherent HEK293 host cells were seeded at about 1×10⁴ viable cells (vc) per cm² of growth area, and grown in cell culture media with 5% CO₂ overlay for 4 days (˜96 hours). Suspension HEK293 host cells were seeded at about 0.2×10⁶ to about 1.0×10⁶ vc per ml of growth area, and grown in cell culture media with 5% CO₂ overlay for 3 days (˜72 hours).

Spent growth media was removed prior to transfection with a polyethylenimine (PEI) transfection reagent (PEIpro® transfection reagent). A plasmid molar ratio of Ad2 helper plasmid to plasmid encoding Rep and Cap polypeptides to plasmid encoding payload of ˜10.8:˜8.2:1 was used for adherent HEK293 cells. A plasmid molar ratio of Ad2 helper plasmid to plasmid encoding Rep and Cap polypeptides to plasmid encoding payload of ˜2:1:0.5 was used for suspension HEK293 cells.

Complexing of PEI transfection reagent and DNA was performed for less than 10 minutes. Transfection complexes were then diluted in production media and added to the cell culture. No FBS was included in the production media.

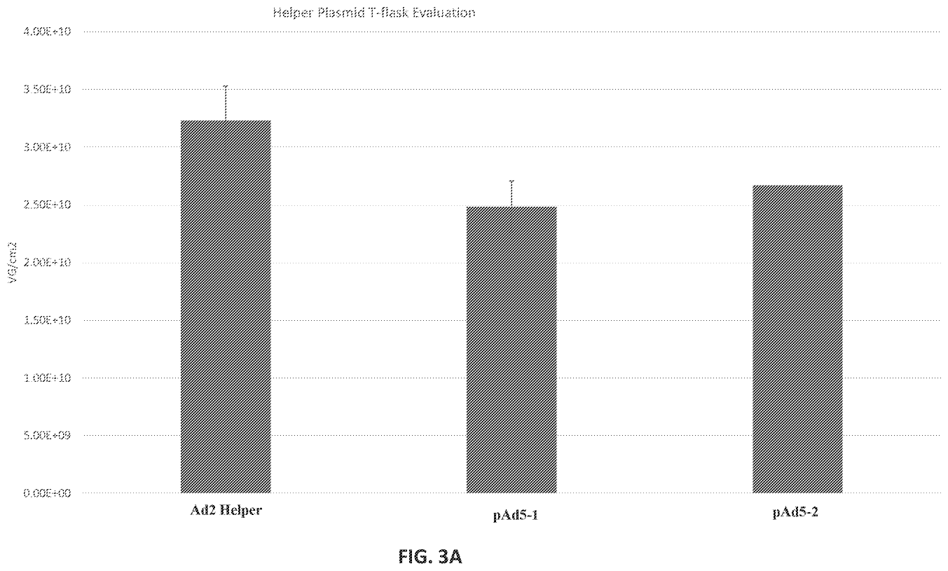

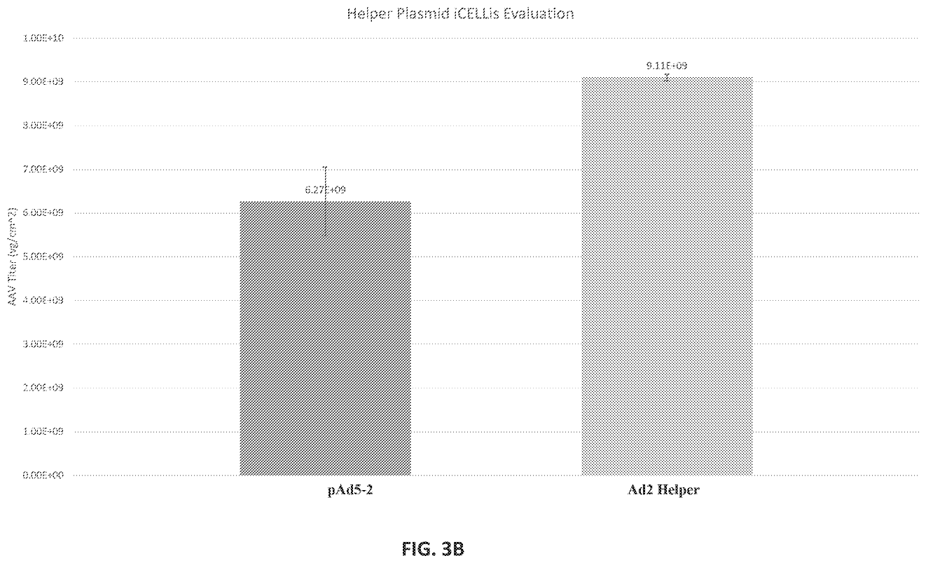

AAV titer or the number of viral genomes (vg)/cm² for the Ad2 helper plasmid exceeded both pAd5-1 and pAd5-2 in t-flask and pAd5-2 in an iCELLis® Nano Bioreactor for adherent cells. In t-flask, use of the Ad2 helper plasmid resulted in over 3.0×10¹⁰ viral genomes (vg)/cm² produced, while use of pAd5-1 and pAd5-2 each resulted in approximately 2.5×10¹⁰ vg/cm² (FIG. 3A). In an iCELLis® Nano Bioreactor, use of the Ad2 helper plasmid resulted in 9.11×10⁹ vg/cm² produced, while use of pAd5-2 resulted in 6.27×10⁹ vg/cm² (FIG. 3B). These results demonstrate that the Ad2 helper plasmid had improved productivity in both t-flask and an iCELLis® Nano Bioreactor compared to Ad5 helper plasmids for adherent cells.

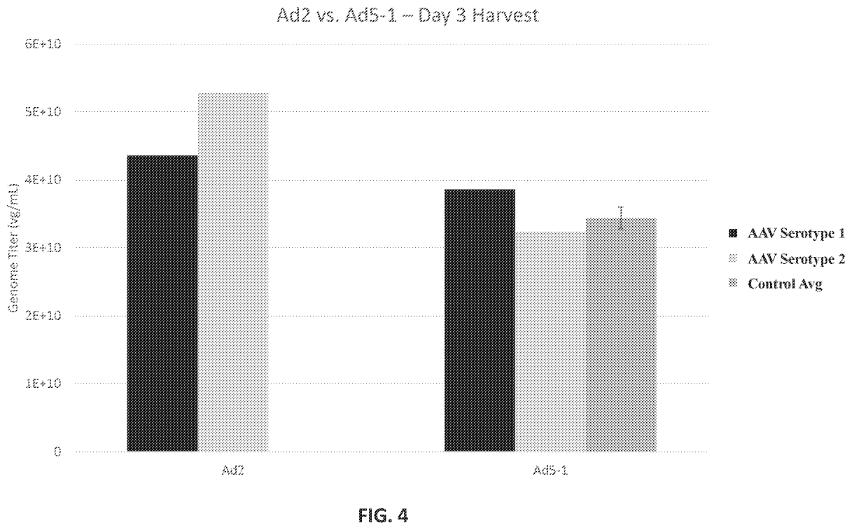

AAV titer or the number of vg/ml for the Ad2 helper plasmid exceeded pAd5-1 in shaker flasks for suspension cells. In shaker flask, use of the Ad2 helper plasmid resulted in over 4.0×10¹⁰ vg/ml to over 5.0×10¹⁰ vg/ml produced, while use of pAd5-1 resulted in approximately 3.0×10¹⁰ vg/cm² to less than 4.0×10¹⁰ vg/ml produced (FIG. 4). These results demonstrate that the Ad2 helper plasmid had improved productivity in shaker flasks compared to an Ad5 helper plasmid for suspension cells.

We claim:

1. A method of producing a plurality of recombinant adeno-associated virus (rAAV) particles comprising:
(a) transfecting a host cell with
(i) a first vector encoding at least one Adenovirus 2 (Ad2) helper polypeptides;
(ii) a second vector encoding at least one Rep polypeptide and/or at least one Cap polypeptide; and
(iii) a third vector encoding at least one payload; and
(b) culturing the host cell under conditions suitable for production of the plurality of rAAV particles,
thereby producing the plurality of rAAV particles.

2. The method of claim 1, wherein the Ad2 helper polypeptides comprise at least one of E1, E2A, E4, or VA RNA.

3. The method of claim 2, wherein the Ad2 helper polypeptides comprise at least two of E1, E2A, E4, or VA RNA.

4. The method of claim 3, wherein the Ad2 helper polypeptides comprise at least three of E1, E2A, E4, or VA RNA.

5. The method of claim 4, wherein the Ad2 helper polypeptides comprise all four of E1, E2A, E4, or VA RNA.

6. The method of any of claims 1-5, wherein the Cap polypeptide comprises an AAV1, AAV2, AAV3, AAV4, AAV5, AAV6, AAV7, AAV8, AAV9, AAV10, or AAV11 Cap polypeptide, or a variant of any of the foregoing.

7. The method of any one of claims 1-6, wherein the Rep polypeptide comprises an AAV2 Rep polypeptide, or a variant thereof.

8. The method of any one of claims 1-7, wherein the host cells are or comprise adherent cells.

9. The method of any one of claims 1-7, wherein the host cells are or comprise suspension cells.

10. The method of any one of claims 1-9, wherein the plurality of rAAV particles are produced in a large-scale preparation.

11. The method of claim 10, wherein the large-scale preparation is at least 10 liters+/−15% of culture media.

12. The method of claim 10, wherein the large-scale preparation is at least 5 m²+/−15% of culture media.

13. The method of any one of claims 1-12, wherein the host cells are transfected using polyethylenimine (PEI).

14. The method of claim 13, wherein transfection complexes are formed for less than 10 minutes+/−15%.

15. The method of any one of claims 1-14, wherein the host cells were seeded prior to the transfecting step at a density of: at least 1.0×10⁴ viable cells (vc)/cm²+/−15%, or at least 1.0×10⁵ vc/cm²+/−15%.

16. The method of any one of claims 1-15, wherein the host cell is a mammalian cell.

17. The method of claim 15, wherein the mammalian cell is selected from HEK293 cells, CHO-K, or HeLa cells.

18. The method of any one of claims 1-17, wherein the host cell expresses an E1 polypeptide.

19. The method of any one of claims 1-18, wherein the plurality of AAV particles are produced at a high titer.

20. The method of claim 19, wherein the high titer is relative to AAV particles produced from an Ad5 helper vector under otherwise identical conditions.

21. The method of claim 19 or 20, wherein the high titer is greater than:
(i) 6.3×10⁹ viral genomes (vg)/cm²+/−15% when cultured in an adherent culture, e.g., a fixed bed bioreactor or a flask;
(ii) 2.6×10¹⁰ vg/cm²+/−15% when cultured in a flask; and/or
(ii) 7.0×10⁹ vg/mL+/−15% when cultured in suspension.

22. The method of any of claims 1-21, further comprising lysis of the host cells.

23. The method of any one of claims 1-22, wherein a lower amount of the first vector is required for production of the plurality of rAAV particles, relative to the amount of an Ad5 helper vector required for production of a plurality of rAAV particles.

24. The method of any one of claims 1-23, wherein the rAAV particles have at least one of the following characteristics:
(a) improved infectivity, relative to rAAV particles produced with another helper vector,
(b) increased expression of the at least one payload, relative to rAAV particles produced with another helper vector, or
(c) improved transduction, relative to rAAV particles produced with another helper vector.

25. The method of any one of claims 1-24, wherein the plurality of rAAV particles is substantially free of one or both of a helper adenovirus or a herpes virus.

26. The method of any one of claims 1-25, wherein the plurality of rAAV have a reduced level of adenoviral impurities relative to rAAV particles produced with an Ad5 helper vector.

27. The method of any one of claims 1-26, wherein the first vector lacks a nucleic acid sequence encoding a Fiber protein.

28. The method of any one of claims 1-27, wherein the third vector further comprises one or both of a sequence encoding a 5′ inverted terminal repeat (ITR) or a sequence encoding a 3′ ITR.

29. The method of claim 28, wherein the first vector comprises the nucleic acid sequence of SEQ ID NO: 1.

30. The method of any one of claims 1-29, wherein the Ad2 helper polypeptides are oriented in the same direction.

31. The method of any one of claims 1-30, wherein the first vector further comprises a nucleic acid sequence of a kanamycin resistance (KanR) gene.

32. The method of any one of claims 1-31, wherein a higher yield of the plurality of rAAV particles is produced relative to a plurality of rAAV particles produced with a helper vector comprising a nucleic acid sequence of an antibiotic resistance gene other than KanR.

33. The method of any one of claims 1-32, further comprising one or both of isolating or purifying the rAAV particles from the host cell.

34. A composition comprising the plurality of rAAV particles formed by the method of any one of claims 1-33.

35. A pharmaceutical composition comprising the composition of claim 34 and a pharmaceutically acceptable component.

36. A method of delivering a gene therapy to a cell or tissue, comprising:
contacting the cell or tissue with the composition of claim 34 or the pharmaceutical composition of claim 35,
thereby delivering the gene therapy to the cell or tissue.

37. A method of treating a subject, the method comprising:
administering the composition of claim 34 or the pharmaceutical composition of claim 35 to the subject,
thereby treating the subject.

38. A reaction mixture comprising:
(i) a first vector encoding at least one Ad2 helper polypeptides;
(ii) a second vector encoding at least one Rep polypeptide and/or at least one Cap polypeptide;
(iii) a third vector encoding a payload; and
(iv) a transfection reagent.

39. A reaction mixture comprising:
(i) a first vector encoding at least one Ad2 helper function polypeptides and at least one Rep polypeptide;
(ii) a second vector encoding at least one Cap polypeptide and at least one payload, and
(iii) a transfection reagent.

40. A transfection complex comprising:
(i) a first vector encoding at least one Ad2 helper polypeptides; and
(ii) a transfection reagent.

41. The transfection complex of claim 40, further comprising one or both of:
(iii) a second vector encoding at least one Rep polypeptide and/or at least one Cap polypeptide); or
(iv) and a third vector encoding at least one payload.

42. The transfection complex of claim 40 or 41, wherein the first vector comprises the nucleic acid sequence of SEQ ID NO: 1.

43. The transfection complex of any of claims 40-42, wherein the nucleic acid sequences encoding the Ad2 helper polypeptides are oriented in the same direction.

44. The transfection complex of claim 43, wherein the Ad2 helper polypeptides comprise VA RNA, E4, and E2A oriented from 5′ to 3′ in direction.

45. A transfection complex comprising:
(i) a first vector encoding at least one Ad2 helper function polypeptides and at least one Rep polypeptide;
(ii) a second vector encoding at least one Cap polypeptide and at least one payload, and
(iii) a transfection reagent.

46. A culture comprising a plurality of host cells and the reaction mixture of any of claim 38 or 39, or the transfection complex of any one of claims 40-45.

47. A bioreactor comprising the culture of claim 46.

48. The bioreactor of claim 47, comprising at least one of:
(i) at least 1×10³ host cells;
(ii) at least 10 liters of culture media; or
(iii) at least 5 m² of culture media m².

49. The bioreactor of claim 47 or 48, wherein one or both of:
(i) the bioreactor is selected from a continuous flow bioreactor, a batch process bioreactor, a perfusion bioreactor, and a fed batch bioreactor; or
(ii) the bioreactor is held under conditions suitable for formation of a plurality of rAAV particles.
